(19) United States
(12) Patent Application Publication
(10) Pub. No.: US 2026/0315645 A1
(43) Pub. Date: Oct. 15, 2026

(54) POLYVALENT POLYMERS AS THERAPEUTICS AND DRESSINGS FOR TISSUE REPAIR, WOUND HEALING, AND ADJUVANTS FOR VACCINE DELIVERY

(71) Applicants: Kaushal Rege, Gilbert, AZ (US); Sudhakar Godeshala, Tempe, AZ (US); Jordan Yaron, Scottsdale, AZ (US)

(72) Inventors: Kaushal Rege, Gilbert, AZ (US); Sudhakar Godeshala, Tempe, AZ (US); Jordan Yaron, Scottsdale, AZ (US)

(73) Assignee: Arizona Board of Regents on behalf of Arizona State University, Scottsdale, AZ (US)

(21) Appl. No.: 19/648,580

(22) Filed: Apr. 15, 2026

Related U.S. Application Data
(60) Provisional application No. 63/789,015, filed on Apr. 15, 2025.

(51) Int. Cl. A61L 15/22; A61L 15/44; C08L 5/08

(52) U.S. Cl. CPC A61L 15/225; A61L 15/44; C08L 5/08; A61L 2300/214; A61L 2300/236

(57) ABSTRACT

The present disclosure provides compositions and functionalized wound dressings useful for treating wounds, as well as methods of using the same to treat wounds. The present disclosure further provides compositions and methods for inducing an immune response in a subject.

### REFERENCE TO RELATED APPLICATIONS

This application claims priority to U.S. Provisional Application No. 63/789,015, filed Apr. 15, 2025, the contents of which are hereby incorporated by reference in their entirety.

### GOVERNMENT RIGHTS

This invention was made with government support under R01 AR074627 awarded by the National Institutes of Health. The government has certain rights in the invention.

### BACKGROUND

Wound healing is a complex biological process comprised of four phases: hemostasis, inflammation, proliferation, and remodeling (Pastar, I., et al., 2014, Advances in Wound Care, 3(7):445-464; MacLeod, A. S., et al., 2016, Advances in Wound Care, 5(2):65-78; Ghosh, D., et al., 2020, ACS Applied Materials & Interfaces, 12(25):27951-27960). This complex process is a barrier restoration which involves keratinocytes, fibroblasts, and immune cells at early and late stages of healing (Eming, S. A., et al., 2014, Science Translational Medicine, 6(265):265sr6; Wilkinson, H. N., et al., 2020, 10(9):200223; Cialdai, F., et al., 2022, Frontiers in Bioengineering and Biotechnology, 10:958381). During the early stages of wound healing, keratinocytes are activated and migrate to the wound surface. Inflammation plays a crucial role in the process of wound healing, which helps to clear the wound of cell debris and potential pathogens (Hart, J., 2002, Journal of Wound Care, 11(6):205-209; Holzer-Geissler, J. C. J., et al., 2022, Biomedicines, 10(4):856; Velnar, T., et al., 2009, Journal of International Medical Research, 37(5):1528-1542; Eming, S. A., et al., 2007, Journal of Investigative Dermatology, 127(3):514-525; Koh, T. J., et al., 2011, Expert Reviews in Molecular Medicine, 13:e23). During the tissue repair process, the repair mechanism is initiated by recruitment of neutrophils and followed by macrophages, which in turn releases chemical signals, namely cytokines and growth factors (Ellis, S., et al., 2018, Current Dermatology Deports, 7(4):350-358; Ng, M. F., 2010, International Wound Journal, 7(1):55-61; Martin, P., et al., 2005, Trends in Cell Biology, 15(11):599-607; Butterfield, T. A., et al., 2006, Journal of Athletic Training, 41(4):457-465; Soliman, A. M., et al., 2022, International Journal of Molecular Science, 24(1):641). Delay in the wound repair may be caused by interference of many factors at any of the healing stages which includes excessive and prolonged inflammation (Kaur, T., et al., 2022, Biomaterials Advances, 143:213163; Guo, S., et al., 2016, Journal of Dental Research, 89(3):219-229; Landen, N. X., et al., 2016, Cellular and Molecular Life Sciences, 73(20):3861-3885). A minor injury may progress into a chronic wound due to failure in the timely and orderly progression of the healing process (Ghosh, D., et al., 2023, Bioengineering & Translational Medicine, 8(2):e10412; Frykberg, R. G., et al., 2015, Advances in Wound Care, 4(9):560-582). The temporal delivery of therapeutic bioactives can improve wound healing in both acute and diabetic wounds. This may help the delivery of therapeutics to modulate individual stages of wound healing and reduce the need for multiple dosing of therapeutics (Albahri, G., et al., 2023, Life, 13(2):13020317; Weng, T., et al., 2022, Burns & Trauma, 20:tkab049; Zhong, Y., et al., 2024, ACS Omega, 9(30):32268; Li, R., et al., 2022, Advances in Science, 9(10):e2105152; Yaron, J. R., et al., 2024, Frontiers in Immunology, 15:1340405; Zhang, L., et al., 2019, Journal of Clinical Medicine, 8(10):1626).

Current methods for chronic wound care (e.g., for diabetic wounds or other long-term wounds) include topical antibiotics, dressings, growth factor injections, and skin substitutes. However, these methods are often either costly or fail to achieve proper healing. For decades, conventional wisdom has held that growth factors are the primary drivers of wound healing; thus, they have become the focus of therapeutic development. Becaplermin (recombinant human PDGF-BB) is an example of an FDA-approved growth factor therapy. Still, it only shows modest efficacy, is costly, and has the potential to cause cancer in patients.

Accordingly, there remains a need for effective means of wound care. The present disclosure addresses this unmet need.

### SUMMARY OF THE DISCLOSURE

In some aspects, the present disclosure provides compositions comprising a polyvalent polymer, wherein the polyvalent polymer comprises at least one small molecule conjugated to a polymer backbone, wherein the at least one small molecule is selected from the group consisting of histamine, histidine, and variants and derivatives thereof. In some embodiments, the polymer backbone is a hyaluronic acid (HA) backbone. In some embodiments, between about 25% and about 35% of all disaccharide monomers in the HA backbone are conjugated to a small molecule.

In some embodiments, the HA is a high-molecular-weight (HMW) HA. In some embodiments, the HMW HA has an average molecular weight between about 1 and about 1.5 MDa. In some embodiments, the polyvalent polymer has a polydispersity index of less than about 1. In some embodiments, the hydrodynamic diameter of the polymer is between about 1,200 nm and about 1,500 nm. In some embodiments, the zeta potential of the polymer is between about −11 mV and about −14 mV. In some embodiments, the composition comprises an aqueous solution of the polyvalent polymer in a concentration of between about 20 mg/mL and about 100 mg/mL.

In some embodiments, the composition is a gel or a hydrogel. In some embodiments, the composition further comprises at least one additional agent. In some embodiments, the at least one additional agent is selected from the group consisting of an antibiotic agent, an antiviral agent, an antifungal agent, an antiparasitic agent, an analgesic agent, an anesthetic agent, an anti-inflammasome agent, a pro-healing bioactive, an antioxidant, and/or a combination thereof.

In some aspects, the present disclosure provides a functionalized wound dressing comprising a wound dressing saturated or impregnated with a composition of the present invention, wherein, when applied to a wound, the functionalized wound dressing releases the polyvalent polymer into the wound. In some embodiments, the wound dressing is in the form of a bandage, gauze, or patch. In some embodiments, the bandage, gauze, or patch is biodegradable.

In some embodiments, the wound dressing comprises a material selected from the group consisting of a natural polymer and a synthetic polymer. In some embodiments, the natural polymer is at least one selected from the group consisting of cellulose, starch, pectin, gelatin, chitosan, collagen, latex, alginate, carrageenan, silk fibroin, keratin, and/or a combination thereof.

In some embodiments, the synthetic polymer is selected form the group consisting of polyethylene (PE), polysulfone (PS), polypropylene (PP), polylactic acid (PLA), poly(lactide-co-glycolide) (PLGA), polyglycoliyc acid (PGA), polycaprolactone (PCL), polyvinyl alcohol (PVA), polyvinyl chloride (PVC), polyurethane, polyvinylpyrrolidone (PVP), ethyl cellulose, methyl cellulose, nitrocellulose, poly(methacrylate-isobutene-monoisopropylmaleaste), polytetrafluoroethylene (PTFE), polyvinylidene fluoride (PVF), polyacrylate, and/or a combination thereof.

In some embodiments, the composition is a vaccine composition, wherein the at least one additional agent comprises at least one antigenic small molecule, glycan, protein, nucleic acid, virus, fragment of a virus, organism, fragment of an organism, or combination thereof.

In some aspects, the present disclosure provides a method of treating a wound in a tissue comprising: a) applying to the wound: i) a composition of the present invention and a wound dressing; or ii) a functionalized wound dressing of the present invention; and b) allowing the wound to heal with the immunomodulatory agent and wound dressing. In some embodiments, the method further comprises using a suture, staple, clamp, adhesive, or other structural element with the wound dressing or functionalized wound dressing to fasten the wound dressing or functionalized wound dressing to the tissue.

In some embodiments, the method further comprises removing the wound dressing or functionalized wound dressing from the wound and applying a second wound dressing to the wound with or without applying a second administration of the composition of the present invention, or applying a second functionalized wound dressing to the wound.

In some embodiments, the wound is an acute wound or a diabetic wound.

In some aspects, the present disclosure provides a method of inducing an immune response in a subject comprising administering to the subject the composition of the present invention.

### BRIEF DESCRIPTION OF THE FIGURES

For the purpose of illustrating the disclosure, the drawings depict certain embodiments of the disclosure. Those of ordinary skill in the art will understand that the embodiments specifically described herein and illustrated in the accompanying drawings are non-limiting exemplary embodiments and that the scope of the various embodiments of the present disclosure is defined solely by the claims. The features illustrated or described in connection with one exemplary embodiment may be combined with the features of other embodiments. Such modifications and variations are to be included within the scope of the present disclosure.

FIG. 1A through FIG. 1D depict representative images depicting the preparation and characterization of hyaluronic acid (HA) polymers. FIG. 1A depicts a schematic representation of the synthesis of histamine-conjugated HA (HAA). FIG. 1B depicts a schematic representation of the synthesis of rhodamine B-conjugated HAA (HAAR). FIG. 1C depicts a representative ¹H nuclear magnetic resonance (NMR) spectrum of histamine-conjugated HA (HAA). FIG. 1D depicts representative Fourier transform infrared spectroscopy (FT-IR) spectra for HA, HAA, and histidine-conjugated hyaluronic acid (HAD), with CO—NH stretching shown in the dashed box.

FIG. 2A and FIG. 2B depict representative NMR spectra of rhodamine-conjugated HA polymers. FIG. 2A depicts a representative NMR spectrum of HAAR with aromatic peaks highlighted in a dashed box. FIG. 2B depicts a representative NMR spectrum of rhodamine-conjugated HAD (HADR) with aromatic peaks highlighted in a dashed box.

FIG. 3 depicts representative FT-IR spectra of HA and rhodamine-conjugated HA with CO—O ester stretching around 1,500-1,600 cm⁻¹, as shown in the dashed box.

FIG. 4 depicts a representative quantification of molecular weights of HA polymers using gel permeation chromatography (GPC). LMW=low molecular weight, MMW=medium molecular weight, HMW=high-molecular-weight.

FIG. 5A and FIG. 5B depict representative results of HA-polymer activation of J77-Dual macrophages. FIG. 5A depicts representative NF-κB activation in J774-Dual macrophages treated with HA-polymers. FIG. 5B depicts representative IRF3 activation in J774-Dual macrophages treated with HA-polymers. UT=untreated (negative control), LPS=lipopolysaccharide (positive control), cGAMP=cyclic guanosine monophosphate-adenosine monophosphate (positive control).

FIG. 6A and FIG. 6B depict representative cell viability of cells treated with HA polymers by MTT assay. FIG. 6A depicts the representative viability of HaCaT cells treated with HA polymers. FIG. 6B depicts the representative viability of J774-Dual macrophages treated with HA polymers.

FIG. 7A through FIG. 7E depict representative cytokine release of J774-Dual macrophages. FIG. 7A depicts representative IL-1β release by J774-Dual macrophages treated with HA polymers. FIG. 7B depicts representative TNF-α release by J774-Dual macrophages treated with HMW HA polymers. FIG. 7C depicts representative IL-6 release by J774-Dual macrophages treated with HMW HA polymers. FIG. 7D depicts representative IL-4 release by J774-Dual macrophages treated with 100 nM HMW HA polymers. FIG. 7E depicts representative IL-10 release by J774-Dual macrophages treated with HMW HA polymers. For FIG. 7A through FIG. 7E, data are presented as mean±SD, n=3, LPS=lipopolysaccharide (positive control).

FIG. 8 depicts representative confocal microscopy images of HaCaT cells (top) and J775-Dual macrophages (bottom) after 24 hours of incubation with rhodamine-conjugated HA (HAR, left), rhodamine-conjugated HAA (HAAR, center), and rhodamine-conjugated HAD (HADR, right).

FIG. 9A through FIG. 9F depict representative results of calcium flux analysis in HaCaT cells treated with HA polymers. FIG. 9A depicts representative results of HA polymer screening for calcium flux with an HRH1 agonist, 2-pyridylethylamine, as a positive control. FIG. 9B depicts representative baseline calcium flux in HaCaT cells. FIG. 9C depicts representative calcium flux in HaCaT cells treated with 100 μM histamine. FIG. 9D depicts representative calcium flux in HaCaT cells treated with 100 μM HAD. FIG. 9E depicts representative calcium flux in HaCaT cells pre-treated with inhibitors (HRHlinh through HRH4inh) of histamine receptor 1 (HRH1) through histamine receptor 4 (HRH4) and subsequently treated with 100 μM HAD. FIG. 9F depicts a representative quantitative analysis of calcium flux depicted in FIG. 9B through FIG. 9E.

FIG. 10A and FIG. 10B depict representative effects of HA polymers on keratinocyte migration. FIG. 10A depicts a representative quantification of wound area for HaCaT cells scratched and treated with HA-HMW, three batches of HAA-HMW (HAA1, HAA2, and HAA3), and three batches of HAD-HMW (HAD1, HAD2, and HAD3) compared to untreated (negative control) and TGF-β-treated (positive control) cells. FIG. 10B depicts representative images of scratch closure for wounds as treated in FIG. 10A.

FIG. 11A and FIG. 11B depict representative results of wound closure experiments with BALB/c mice treated with HA polymers. FIG. 11A depicts representative images of wound closure of BALB/c mice 0, 4, and 8 days after treatment. FIG. 11B depicts representative results of daily planimetry analysis of full-thickness splinted wounds (5 mm initial diameter) after single treatment with saline (negative control), HA-HMW, HAA-HMW, and HAD-HMW.

FIG. 12A through FIG. 12C depict representative effects of HA polymers on excisional wound closure in BALB/c mice. FIG. 12A depicts representative images of tissue sections excised eight days after treatment of wounds in BALB/c mice treated with HA polymers stained with hematoxylin and eosin (H&E). Arrows indicate the edges of the epidermal gap, and dotted lines indicate the granulation area. FIG. 12B depicts a representative quantification of the epidermal gap of excisional wounds eight days after treatment with HA polymers. FIG. 12C depicts a representative quantification of the granulation area of excisional wounds eight days after treatment with HA polymers.

FIG. 13A through FIG. 13D depict representative effects of HA polymers on immune response in excisional wounds of BALB/c mice. FIG. 13A depicts representative images of excised wound tissue sections treated with HA polymers stained by immunohistochemistry for Ym-1, Arginase-1, and CD86. FIG. 13B depicts a representative quantification of Ym-1⁺ cells treated with HA polymers. FIG. 13C depicts a representative quantification of Arginase-1⁺ cells treated with HA polymers. FIG. 13D depicts a representative quantification of CD86⁺ cells treated with HA polymers.

FIG. 14A through FIG. 14D depict representative effects of HA polymers on non-immune response in excisional wounds of BALB/c mice. FIG. 14A depicts representative images of excised wound tissue sections treated with HA polymers stained by immunohistochemistry for PCNA, α-SMA, CD31, and E-cadherin. FIG. 14B depicts a representative quantification of keratinocytes positive for nuclear PCNA eight days after treatment with HA polymers. FIG. 14C depicts a representative quantification of α-SMA⁺ cells eight days after treatment with HA polymers. FIG. 14D depicts a representative quantification of E-cadherin loss in keratinocytes eight days after treatment with HA polymers.

### DETAILED DESCRIPTION

The present disclosure is based, in part, on the unexpected discovery that application of histamine or histidine conjugated to a polymer (e.g., hyaluronic acid (HA)) increases the inflammatory response and promotes wound healing. Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways. Accordingly, in some aspects, the present disclosure is directed to compositions comprising histamine, histidine, or a variant or derivative thereof conjugated to an HA polymer backbone. In some embodiments, a defined percentage of disaccharide monomers in the HA backbone is conjugated to histamine, histidine, or a variant or derivative thereof. In some embodiments, the HA is a high-molecular-weight HA (HA-HMW). In some embodiments, compositions of the disclosure further comprise at least one additional agent. In some embodiments, the at least one additional agent is at least one of an antibiotic agent, an antiviral agent, an antifungal agent, an antiparasitic agent, an anti-inflammatory agent, an analgesic agent, and an anesthetic agent.

In some aspects, the present disclosure is related to a functionalized wound dressing. In some embodiments, the functionalized wound dressing comprises a wound dressing impregnated or saturated with a composition of the present disclosure. In some embodiments, the functionalized wound dressing is a bandage, gauze, or patch. In some embodiments, the functionalized wound is biodegradable.

In another aspect, the present disclosure provides methods of treating a wound. In some embodiments, the method comprises administering to the wound a composition of the present disclosure and applying a wound dressing, or applying a functionalized wound dressing of the present disclosure, to the wound. In some embodiments, the method further comprises replacing the wound dressing with or without reapplying a composition of the disclosure or replacing the functionalized wound dressing.

### Definitions

Unless defined otherwise, all technical and scientific terms used herein have the same meaning as commonly understood by one of ordinary skill in the art to which the disclosure pertains. Although any methods and materials similar or equivalent to those described herein can be used in the practice for testing of the present disclosure, the preferred materials and methods are described herein. In describing and claiming the present disclosure, the following terminology can be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

As used herein, the term “about” can be understood by persons of ordinary skill in the art and will vary to some extent depending on the context in which it is used. As used herein when referring to a measurable value such as an amount, a temporal duration, and the like, the term “about” is meant to encompass variations of ±20%, ±10%, ±5%, ±1%, or ±0.1% from the specified value, as such variations are appropriate to perform the disclosed methods.

As used herein, the term “alkyl,” by itself or as part of another substituent, means, unless otherwise stated, a straight or branched chain hydrocarbon having the number of carbon atoms designated (i.e., C₁₋₆ means one to six carbon atoms) and includes straight, branched chain, cyclic, and substituent groups, optionally substituted. Examples include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. The term “alkyl,” unless otherwise noted, is also meant to include those derivatives of alkyl defined in more detail below, such as “heteroalkyl,” “haloalkyl,” and “homoalkyl.”

As used herein, the term “alkenyl,” by itself or as part of another substituent means, unless otherwise stated, a straight or branched chain hydrocarbon having the number of carbon atoms designated (i.e. C₂₋₆ means one to six carbon atoms) including at least one carbon-carbon double bond, and includes straight, branched chain, or cyclic substituent groups, optionally substituted.

As used herein, the term “alkynyl,” by itself or as part of another substituent means, unless otherwise stated, a straight or branched chain hydrocarbon having the number of carbon atoms designated (i.e. C₃₋₆ means one to six carbon atoms) including at least one carbon-carbon triple bond, and includes straight, branched chain, or cyclic substituent groups, optionally substituted.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with at least one additional group(s) individually and independently selected from groups described herein.

In one embodiment, the substituents are independently selected from oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoroalkyl, substituted or unsubstituted heteroalkyl, substituted or unsubstituted alkoxy, fluoroalkoxy, —S-alkyl, S(═O)₂alkyl, —C(═O)NH[substituted or unsubstituted alkyl, or substituted or unsubstituted phenyl], —C(═O)N[H or alkyl]₂, —OC(═O)N[substituted or unsubstituted alkyl]₂, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH)[substituted or unsubstituted alkyl]₂, or —C(NH₂)[substituted or unsubstituted alkyl]₂. In another embodiment, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, —ON(O)₂, or —C(═O)OH. In yet one embodiment, the substituents are independently selected from C₁₋₆ alkyl, —OH, C₁₋₆ alkoxy, halo, amino, acetamido, oxo, or nitro. In yet another embodiment, the substituents are independently selected from C₁₋₆ alkyl, C₁₋₆ alkoxy, halo, acetamido, or nitro. As used herein, where a substituent is an alkyl or alkoxy group, the carbon chain may be branched, straight, or cyclic.

As used here, “biocompatible” refers to any material that, when implanted in a mammal, does not provoke an adverse response in the mammal. A biocompatible material, when introduced into an individual, is not toxic or injurious to that individual, nor does it induce immunological rejection of the material in the mammal.

“Hydrogel” refers to a water-insoluble and water-swellable cross-linked polymer that is capable of absorbing at least 3 times, preferably at least 10 times, its own weight of a liquid. “Hydrogel” can also refer to a “thermo-responsive polymer” as used herein.

“Measuring” or “measurement,” or “detecting” or “detection,” or “determine” or “determining” means assessing the presence, absence, quantity, or amount of either a given substance within a clinical or subject-derived sample, including the derivation of qualitative or quantitative concentration levels of such substances.

The terms “patient,” “subject,” “individual,” and the like are used interchangeably herein and refer to any animal, or cells thereof, whether in vitro or in situ, amenable to the methods described herein. In certain non-limiting embodiments, the patient, subject, or individual is a human.

As used herein, the term “pharmaceutical composition” refers to a mixture of at least one compound of the disclosure with other chemical components and entities, such as carriers, stabilizers, diluents, dispersing agents, suspending agents, thickening agents, and/or excipients. The pharmaceutical composition facilitates the administration of the compound to an organism. Multiple techniques of administering a compound exist in the art, including, but not limited to, intravenous, oral, aerosol, parenteral, ophthalmic, pulmonary, and topical administration.

The term “pharmacological composition,” “therapeutic composition,” “therapeutic formulation” or “pharmaceutically acceptable formulation” can mean, but is in no way limited to, a composition or formulation that allows for the effective distribution of an agent provided by the disclosure, which is in a form suitable for administration to the physical location most suitable for their desired activity, e.g., systemic administration. As used herein, the term “pharmaceutical composition” refers to a mixture of at least one compound of the disclosure with other chemical components and entities, such as carriers, stabilizers, diluents, dispersing agents, suspending agents, thickening agents, and/or excipients. The pharmaceutical composition facilitates the administration of the compound to an organism. Multiple techniques for administering a compound exist in the art, including, but not limited to, intravenous, topical, intraperitoneal, intramuscular, oral, aerosol, parenteral, ophthalmic, and pulmonary administration. Non-limiting examples of agents suitable for formulation with the, e.g., compounds provided by the instant disclosure include: cinnamoyl, PEG, phospholipids or lipophilic moieties, phosphorothioates, P-glycoprotein inhibitors (such as Pluronic P85) which can enhance entry of drugs into various tissues, for example the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, D F et al, 1999, Cell Transplant, 8, 47-58) Alkermes, Inc. Cambridge, Mass.; loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999); and oil-based delivery systems (Kirtane et al. 2022, Sci. Adv. 8, eabm8478).

“Pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance, and bioavailability. “Pharmaceutically acceptable carrier” refers to a medium that does not interfere with the effectiveness of the biological activity of the active ingredient(s) and is not toxic to the host to which it is administered.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Typically, such constructs are carried or transported from one organ or body part to another. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the patient. Supplementary active compounds may also be incorporated into the compositions. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example, in Remington's Pharmaceutical Sciences (Genaro, Ed., Mack Publishing Co., 1985, Easton, PA), which is incorporated herein by reference.

As used herein, “polymer” includes copolymers and may be natural, semisynthetic, or synthetic. “Natural” polymers, as used herein, refer to polymers that occur and are present in nature. “Semisynthetic” polymers, as used herein, refer to natural polymers that are modified through chemical or physical treatment. As used herein, “synthetic” polymers refer to polymers that are synthesized artificially by humans. “Copolymers” are polymers formed of more than one polymer precursor and may similarly be natural (i.e., at least two naturally occurring polymers that are combined naturally, e.g., a polysaccharide enzymatically linked to a polypeptide), semisynthetic (i.e., at least two naturally occurring polymers that are artificially combined or a natural copolymer modified through chemical or physical treatment), or synthetic (i.e., a synthetic polymer linked to at least one other synthetic or natural polymer). Polymers as used herein include those that are soluble in a solvent and are insoluble in an antisolvent.

A “therapeutic” treatment is a treatment administered to a subject who exhibits a sign or symptom of pathology, for the purpose of diminishing or eliminating that sign or symptom.

As used herein, the terms “therapy” or “therapeutic regimen” refer to those activities taken to alleviate or alter a disorder or disease state, e.g., a course of treatment intended to reduce or eliminate at least one sign or symptom of a disease or disorder using pharmacological, surgical, dietary, and/or other techniques. A therapeutic regimen may include a prescribed dosage of at least one drug or surgery. Therapies will most often be beneficial and reduce or eliminate at least one sign or symptom of the disorder or disease state. Still, in some instances, a therapy will have undesirable side effects. The effect of therapy will also be influenced by the subject's physiological state, e.g., age, gender, genetics, weight, and other disease conditions.

The term “therapeutically effective amount” refers to the amount of the subject compound that will elicit the biological or medical response of a tissue, system, or subject that the researcher, veterinarian, medical doctor, or other clinician is seeking. The term “therapeutically effective amount” includes that amount of a compound that, when administered, is sufficient to prevent the development of, or alleviate to some extent, at least one of the signs or symptoms of the disorder or disease being treated. The therapeutically effective amount will vary depending on the compound, the disease, its severity, and the age, weight, and other factors of the subject to be treated.

To “treat” a disease as the term is used herein means to reduce the frequency or severity of at least one sign or symptom of a disease or disorder experienced by a subject.

As used herein, “wound healing” is intended to include all disorders characterized by any disease, disorder, syndrome, anomaly, pathology, or abnormal condition of the skin and/or underlying connective tissue, e.g., skin wounds caused by mechanical injury, skin wounds following surgery, skin abrasions caused by mechanical trauma, caustic agents or burns, cornea following cataract surgery or corneal transplants, mucosal epithelium wounds following infection or drug therapy (e.g., respiratory, gastrointestinal, genitourinary, mammary, oral cavity, ocular tissue, liver and kidney), diabetic wounds, skin wounds following grafting, and regrowth of blood vessels following angioplasty.

Ranges: Throughout this disclosure, various aspects may be presented as ranges. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the range's breadth.

### Compositions

The present disclosure is based, in part, on the unexpected discovery that a polymer (e.g., HA) conjugated to specific nitrogenous small molecules can increase inflammatory responses and promote wound healing. Accordingly, the present disclosure provides a variety of hyaluronic acid conjugates that are useful in various wound-related applications.

In some embodiments, the present disclosure provides a composition comprising a polyvalent polymer, wherein the polyvalent polymer comprises a small molecule conjugated to the polymer backbone. In certain instances, the polymer may comprise a biopolymer (i.e., natural polymer), a synthetic polymer, or a combination thereof. Examples of biopolymers include, but are not limited to, cHA, cellulose, starch, pectin, gelatin, chitosan, collagen, latex, alginate, carrageenan, silk fibroin, and keratin. In some embodiments, the biopolymer is HA. Examples of suitable synthetic polymers include, but are not limited to, polyethylene (PE), polysulfone (PS), polypropylene (PP), polylactic acid (PLA), poly(lactide-co-glycolide) (PLGA), polyglycoliyc acid (PGA), polycaprolactone (PCL), polyvinyl alcohol (PVA), polyvinyl chloride (PVC), polyurethane, polyvinylpyrrolidone (PVP), ethyl cellulose, methyl cellulose, nitrocellulose, poly(methacrylate-isobutene-monoisopropylmaleaste), polytetrafluoroethylene (PTFE), polyvinylidene fluoride (PVF), polyacrylate, polyamide, poly(ethylene oxide) (PEO), poly(glycolic-caprolactone), polyhydroxyalkanoate, polyanhydride, poly(orthocarbonate, polycarbonate, polyphosphonate, polysiloxanes, and functionalized and derivatized versions thereof.

In some embodiments, the small molecules comprise at least one nitrogen. In some embodiments, the small molecules comprise at least two nitrogen atoms. In some embodiments, the small molecule comprises a cyclic component comprising at least two nitrogen atoms. In some embodiments, the small molecule comprises an imidazole ring. In some embodiments, the small molecule is a molecule of Formula (I):

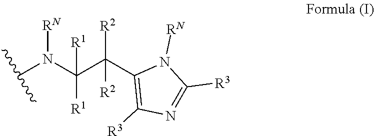

wherein:
each instance of R^{N} is independently selected from the group consisting of H, D, F, methyl, ethyl, propyl, isopropyl, and combinations thereof;
each instance of R¹, and R³ is independently selected from the group consisting of H, D, F, C₁-C₃ alkyl, C₂-C₃ alkenyl, C₂-C₃ alkynyl, carbonyl, carboxyl, amide, ester, ether, and combinations thereof;
each instance of R² is independently selected from the group consisting of H, D, F, C₁-C₃ alkyl, C₂-C₃ alkenyl, C₂-C₃ alkynyl, and combinations thereof; and
the compound of Formula (I) is conjugated to the polymer backbone via an amide bond, as designated by the symbol [symbol] .

In some embodiments, the compound of Formula (I) is histamine, histidine, or a derivative thereof. In some embodiments, the compound of Formula (I) is histamine or histidine. In some embodiments, the small molecule is histamine. In some embodiments, each instance of R^{N}, R¹, R², and R³ is independently H or D. In some embodiments, all of R^{N}, R¹, R², and R³ are H.

In some embodiments, the compound of Formula (I) is histidine. In some embodiments, one R¹ is H or D, one R¹ is carboxyl, and each instance of R^{N}, R², and R³ is independently H or D. In some embodiments, one R¹ is H, one R¹ is —COOH, and all of R^{N}, R², and R³ are H.

In some embodiments, a small molecule of Formula (I) is conjugated to a monomer of the polymer backbone. In some embodiments, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, or at least about 25% of the monomers of the polymer are conjugated to a small molecule of Formula (I).

In some embodiments, the polyvalent polymer comprises HA, wherein a small molecule of Formula (I) is conjugated to a disaccharide monomer of the HA backbone. In some embodiments, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, or at least about 25% of the disaccharide monomers of the HA are conjugated to a small molecule of Formula (I).

In some embodiments, the polyvalent polymer comprises HA conjugated to histamine (HAA). In some embodiments at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, or at least about 25% of the disaccharide monomers of the HAA are conjugated to histamine.

In some embodiments, the polyvalent polymer comprises HA conjugated to histidine (HAD). In some embodiments, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, or at least about 25% of the disaccharide monomers of the HAD are conjugated to histamine.

In some embodiments, between about 1% and about 50%, between about 5% and about 45%, between about 10% and about 40%, between about 15% and about 40%, between about 20% and about 40%, and between about 25% and about 35% of the polymer monomers are conjugated to a small molecule of Formula (I). In some embodiments, about 25%, about 26%, about 27%, about 28%, about 29%, about 30%, about 31%, about 32%, about 33%, about 34%, or about 35% of the polymer monomers are conjugated to a small molecule of Formula (I).

In some embodiments, between about 1% and about 50%, between about 5% and about 45%, between about 10% and about 40%, between about 15% and about 40%, between about 20% and about 40%, and between about 25% and about 35% of the disaccharide monomers of the HA are conjugated to a small molecule of Formula (I). In some embodiments, about 25%, about 26%, about 27%, about 28%, about 29%, about 30%, about 31%, about 32%, about 33%, about 34%, or about 35% of the disaccharide monomers of the HA are conjugated to a small molecule of Formula (I).

In some embodiments, the compound is HAA. In some embodiments, between about 1% and about 50%, between about 5% and about 45%, between about 10% and about 40%, between about 15% and about 40%, between about 20% and about 40%, and between about 25% and about 35% of the disaccharide monomers of the HAA are conjugated to histamine. In some embodiments, about 25%, about 26%, about 27%, about 28%, about 29%, about 30%, about 31%, about 32%, about 33%, about 34%, or about 35% of the disaccharide monomers of the HAA are conjugated to histamine.

In some embodiments, the compound is HAD. In some embodiments, between about 1% and about 50%, between about 5% and about 45%, between about 10% and about 40%, between about 15% and about 40%, between about 20% and about 40%, and between about 25% and about 35% of the disaccharide monomers of the HAD are conjugated to histidine. In some embodiments, about 25%, about 26%, about 27%, about 28%, about 29%, about 30%, about 31%, about 32%, about 33%, about 34%, or about 35% of the disaccharide monomers of the HAD are conjugated to histidine.

In some embodiments, the polyvalent polymer is a low-molecular-weight (LMW), medium-molecular-weight (MMW), or high-molecular-weight (HMW) polymer. In some embodiments, the polyvalent polymer comprises low-molecular-weight HA (LMW-HA), medium-molecular-weight HA (MMW-HA), or high-molecular-weight HA (HMW-HA) conjugated to a small molecule of Formula (I). In some embodiments, the polyvalent polymer comprises HAA or HAD. In some embodiments, the HAA comprises LMW-HA (LMW-HAA), MMW-HA (MMW-HAA), or HMW-HA (HMW-HAA). In some embodiments, the HAD comprises LMW-HA (LMW-HAD), MMW-HA (MMW-HAD), or HMW-HA (HMW-HAD).

In some embodiments, the LMW polyvalent polymer has an average molecular weight between about 100 kDa and about 250 kDa, between about 150 kDa and about 250 kDa, or between about 175 kDa and about 225 kDa. In some embodiments, the LMW-HA conjugated to a small molecule of Formula (I), LMW-HAA, or LMW-HAD has an average molecular weight between about 100 kDa and about 250 kDa, between about 150 kDa and about 250 kDa, or between about 175 kDa and about 225 kDa. In some embodiments, the LMW polyvalent polymer has an average molecular weight of about 175 kDa, about 180 kDa, about 185 kDa, about 190 kDa, about 195 kDa, about 200 kDa, about 205 kDa, about 210 kDa, about 215 kDa, about 220 kDa, and about 225 kDa. In some embodiments, the LMW-HA conjugated to a small molecule of Formula (I), LMW-HAA, or LMW-HAD has an average molecular weight of about 175 kDa, about 180 kDa, about 185 kDa, about 190 kDa, about 195 kDa, about 200 kDa, about 205 kDa, about 210 kDa, about 215 kDa, about 220 kDa, and about 225 kDa.

In some embodiments, the MMW polyvalent polymer has an average molecular weight between about 200 kDa and about 500 kDa, between about 250 kDa and about 450 kDa, between about 300 kDa and about 400 kDa, or between about 350 kDa and about 450 kDa. In some embodiments, the MMW-HA conjugated to a small molecule of Formula (I), MMW-HAA, or MMW-HAD has an average molecular weight between about 200 kDa and about 500 kDa, between about 250 kDa and about 450 kDa, between about 300 kDa and about 400 kDa, or between about 350 kDa and about 450 kDa. In some embodiments, the MMW polyvalent polymer has an average molecular weight of about 350 kDa, about 360 kDa, about 370 kDa, about 380 kDa, about 390 kDa, about 400 kDa, about 410 kDa, about 420 kDa, about 430 kDa, about 440 kDa, or about 450 kDa. In some embodiments, the MMW-HA conjugated to a small molecule of Formula (I), MMW-HAA, or MMW-HAD has an average molecular weight of about 350 kDa, about 360 kDa, about 370 kDa, about 380 kDa, about 390 kDa, about 400 kDa, about 410 kDa, about 420 kDa, about 430 kDa, about 440 kDa, or about 450 kDa.

In some embodiments, the HMW polyvalent polymer has an average molecular weight between about 1 MDa and about 5 MDa, between about 1 MDa and about 4 MDa, between about 1 MDa and about 3 MDa, between about 1 MDa and about 2 MDa, or about 1 MDa and about 1.5 MDa. In some embodiments, the HMW-HA conjugated to a small molecule of Formula (I), HMW-HAA, or HMW-HAD has an average molecular weight between about 1 MDa and about 5 MDa, between about 1 MDa and about 4 MDa, between about 1 MDa and about 3 MDa, between about 1 MDa and about 2 MDa, or about 1 MDa and about 1.5 MDa. In some embodiments, the HMW polyvalent polymer has an average molecular weight of about 1 MDa, about 1.05 MDa, about 1.1 MDa, about 1.15 MDa, about 1.2 MDa, about 1.25 MDa, about 1.3 MDa, about 1.35 MDa, about 1.4 MDa, about 1.45 MDa, and about 1.5 MDa. In some embodiments, the HMW-HA conjugated to a small molecule of Formula (I), HMW-HAA, or HMW-HAD has an average molecular weight of about 1 MDa, about 1.05 MDa, about 1.1 MDa, about 1.15 MDa, about 1.2 MDa, about 1.25 MDa, about 1.3 MDa, about 1.35 MDa, about 1.4 MDa, about 1.45 MDa, and about 1.5 MDa

In some embodiments, the polymer has a polydispersity index (PDI) less than about 3, less than about 2.5, less than about 2, less than about 1.5, or less than about 1. In some embodiments, the HA conjugated to a small molecule of Formula (I), HAA, or HAD has a PDI less than about 3, less than about 2.5, less than about 2, less than about 1.5, or less than about 1.

In some embodiment, the polyvalent polymer has an average hydrodynamic diameter between about 500 nm and about 2,500 nm, between about 500 nm and about 2,400 nm, between about 500 nm and about 2,300 nm, between about 500 nm and about 2,200 nm, between about 600 nm and about 2,100 nm, between about 700 and about 2,000 nm, between about 800 nm and about 1,900 nm, between about 900 nm and about 1,800 nm, between about 1,000 nm and about 1,700 nm, between about 1,100 nm and about 1,600 nm, and between about 1,200 nm and about 1,500 nm. In some embodiment, the HA conjugated to a small molecule of Formula (I), HAA, or HAD has an average hydrodynamic diameter between about 500 nm and about 2,500 nm, between about 500 nm and about 2,400 nm, between about 500 nm and about 2,300 nm, between about 500 nm and about 2,200 nm, between about 600 nm and about 2,100 nm, between about 700 and about 2,000 nm, between about 800 nm and about 1,900 nm, between about 900 nm and about 1,800 nm, between about 1,000 nm and about 1,700 nm, between about 1,100 nm and about 1,600 nm, and between about 1,200 nm and about 1,500 nm. In some embodiments, the polyvalent polymer has an average hydrodynamic diameter of about 1,200 nm, about 1,250 nm, about 1,300 nm, about 1,350 nm, about 1,400 nm, about 1,450 nm, or about 1,500 nm. In some embodiments, the HA conjugated to a small molecule of Formula (I), HAA, or HAD has an average hydrodynamic diameter of about 1,200 nm, about 1,250 nm, about 1,300 nm, about 1,350 nm, about 1,400 nm, about 1,450 nm, or about 1,500 nm.

In some embodiments, the polyvalent polymer has a zeta potential between about −5 mV and about −20 mV, between about −6 mV and about −19 mV, between about −7 mV and about −18 mV, between about −8 mV and about −17 mV, between about −9 mV and about −16 mV, between about −10 mV and about −15 mV, or between about −11 mV and about −14 mV. In some embodiments, the HA conjugated to a small molecule of Formula (I), HAA, or HAD has a zeta potential between about −5 mV and about −20 mV, between about −6 mV and about −19 mV, between about −7 mV and about −18 mV, between about −8 mV and about −17 mV, between about −9 mV and about −16 mV, between about −10 mV and about −15 mV, or between about −11 mV and about −14 mV. In some embodiments, the polyvalent polymer has a zeta potential of about −11 mV, about −12 mV, about −13 mV, or about −14 mV. In some embodiments, the HA conjugated to a small molecule of Formula (I), HAA, or HAD has a zeta potential of about −11 mV, about −12 mV, about −13 mV, or about −14 mV.

In some embodiments, a composition of the present disclosure is a solution, gel, hydrogel, cream, lotion, ointment, or other similar formulation. In some embodiments, the composition comprises the polyvalent polymer in a concentration of between about 1 ng/mL and about 1 g/mL, between about 10 ng/mL and about 1 g/mL, between about 20 ng/mL and about 1 g/mL, between about 30 ng/mL and 1 g/mL, between about 40 ng/mL and 1 g/mL, between about 50 ng/mL and 1 g/mL, between about 60 ng/mL and 1 g/mL, between about 70 ng/mL and 1 g/mL, between about 80 ng/mL and about 1 g/mL, between about 90 mg/mL and 1 g/mL, between about 100 ng/mL and about 1 g/mL, between about 200 ng/mL and about 1 g/mL, between about 300 ng/mL and about 1 g/mL, between about 400 ng/mL and about 1 g/mL, between about 500 ng/mL and about 1 g/mL, between about 600 ng/mL and about 1 g/mL, between about 700 ng/mL and about 1 g/mL, between about 800 ng/mL and about 1 g/mL, between about 900 ng/mL and about 1 g/mL, between about 1 mg/mL and about 1 g/mL, between about 2 mg/mL and about 900 mg/mL, between about 3 mg/mL and about 800 mg/mL, between about 4 mg/mL and about 700 mg/mL, between about 5 mg/mL and about 600 mg/mL, between about 6 mg/mL and about 500 mg/mL, between about 7 mg/mL and about 400 mg/mL, between about 8 mg/mL and about 300 mg/mL, between about 9 mg/mL and about 200 mg/mL, between about 10 mg/mL and about 175 mg/mL, between about 11 mg/mL and about 150 mg/mL, between about 12 mg/mL and about 125 mg/mL, between about 13 mg/mL and about 100 mg/mL, between about 14 mg/mL and about 100 mg/mL, between about 15 mg/mL and about 100 mg/mL, between about 16 mg/mL and about 100 mg/mL, between about 17 mg/mL and about 100 mg/mL, between about 18 mg/mL and about 100 mg/mL, between about 19 mg/mL and about 100 mg/mL, or between about 20 mg/mL and about 100 mg/mL.

In some embodiments, the composition comprises HA conjugated to a small molecule of Formula (I), HAA, or HAD in a concentration of between about 1 ng/mL and about 1 g/mL, between about 10 ng/mL and about 1 g/mL, between about 20 ng/mL and about 1 g/mL, between about 30 ng/mL and 1 g/mL, between about 40 ng/mL and 1 g/mL, between about 50 ng/mL and 1 g/mL, between about 60 ng/mL and 1 g/mL, between about 70 ng/mL and 1 g/mL, between about 80 ng/mL and about 1 g/mL, between about 90 mg/mL and 1 g/mL, between about 100 ng/mL and about 1 g/mL, between about 200 ng/mL and about 1 g/mL, between about 300 ng/mL and about 1 g/mL, between about 400 ng/mL and about 1 g/mL, between about 500 ng/mL and about 1 g/mL, between about 600 ng/mL and about 1 g/mL, between about 700 ng/mL and about 1 g/mL, between about 800 ng/mL and about 1 g/mL, between about 900 ng/mL and about 1 g/mL, between about 1 mg/mL and about 1 g/mL, between about 2 mg/mL and about 900 mg/mL, between about 3 mg/mL and about 800 mg/mL, between about 4 mg/mL and about 700 mg/mL, between about 5 mg/mL and about 600 mg/mL, between about 6 mg/mL and about 500 mg/mL, between about 7 mg/mL and about 400 mg/mL, between about 8 mg/mL and about 300 mg/mL, between about 9 mg/mL and about 200 mg/mL, between about 10 mg/mL and about 175 mg/mL, between about 11 mg/mL and about 150 mg/mL, between about 12 mg/mL and about 125 mg/mL, between about 13 mg/mL and about 100 mg/mL, between about 14 mg/mL and about 100 mg/mL, between about 15 mg/mL and about 100 mg/mL, between about 16 mg/mL and about 100 mg/mL, between about 17 mg/mL and about 100 mg/mL, between about 18 mg/mL and about 100 mg/mL, between about 19 mg/mL and about 100 mg/mL, or between about 20 mg/mL and about 100 mg/mL.

In some embodiments, compositions of the present disclosure further comprise at least one additional agent. Examples of additional agents that may be suitable include, but are not limited to, antiviral agents, antibiotic agents, antifungal agents, antiparasitic agents, pain relief agents, analgesic agents, anesthetic agents, anti-inflammasome agents, pro-healing bioactives, and antioxidants.

Examples of suitable antiviral agents include, but are not limited to, nucleoside reverse transcriptase inhibitors (NRTIs, e.g., abacavir, emtricitabine, lamuvidine, tenofovir disoproxil fumarate, zidovudine, entecavir, etc.), non-nucleoside reverse transcriptase inhibitors (NNRTIs, e.g., doravirine, efavirenz, etravirine, nevirapine, rilpivirine, adefovir, etc.), protease inhibitors (PIs, e.g., atazanavir, darunavir, fosamprenavir, ritonavir, tipranavir, grazoprevir, glecaprevir, voxilaprevir, etc.), fusion inhibitors (e.g., enfuvirtide, etc.), CCR5 antagonists (e.g., maraviroc, etc.), integrase strand transfer inhibitors (NSTIs, e.g., cabotegravir, dolutegravir, raltegravir, etc.), attachment inhibitors (e.g., fostemsavir, etc.), post-attachment inhibitors (e.g., ibalizumab-uiyk, etc.), capsid inhibitors (e.g., lenacapavir, etc.), pharmacokinetic enhancers (e.g., cobicistat, etc.), nucleoside analogs (e.g., ribavirin, sofosbuvir, telbivudine, etc.), neuraminidase inhibitors (e.g., oseltamivir, zanamivir, peramivir, etc.), protein inhibitors (e.g., elbasvir, pibrentasvir, ledipasvir, velpatasvir, baloxavir marboxil, etc.).

Examples of suitable antibiotic agents include, but are not limited to, isoniazid, rifampin, pyrazinamide, ethambutol, bedaquiline, linezolid, pretomanid, rifapentine, cycloserine, ampicillin, gentamicin, trimethoprim-sulfamethoxazole, levofloxacin, penicillin, meropenem, vancomycin, and azithromycin.

Examples of suitable antifungal agents include, but are not limited to, clotrimazole, econazole, miconazole, terbinafine, fluconazole, ketoconazole, nystatin, amphotericin, bifonazole, butocanaozle, fenticonazole, isoconazole, luliconazole, miconazole, omoconazole, oxiconazole, sertaconazole, sulconazole, tioconazole, albaconazole, cyproconazole, efinaconazole, epoxiconazole, isavuconazole, itraconazole, posaconazole, propiconazole, ravuconazole, terconazole, voriconazole, abafungin, butenafine, naftinifine, terbinafine, anidulafungin, caspofungin, micafungin, ibrexafungerp, acrisorcin, amorlfine, aurones, benzoic acid, carbol fuchsin, ciclopirox, clioquinol, crystal violet, chlorhexidine, chlorophetanol, diiodohydroxyquinoline, flucytosine, fumagillin, griseofulvin, haloprogin, miltefosine, nikkomycin, orotomide, piroctone olamine, pentanenitrile, tolnaftate, and triacetin. Examples of suitable antiparasitic agents include, but are not limited to, chloroquine, hydroxychloroquine, artemisinin, quinine, primaquine, artesunate, artemether, lumefantrine, atovaquone, proguanil, mefloquine, tetracycline, clindamycin, pyrimethamine, sulfadiazine, atovaquone, spiramycin, leucovorin, miltefosine, amphotericin B, sodium stibogluconate, paromomycin, pentamidine isethionate, ketoconazole, fluconazole, and meglumine antimonate.

Examples of suitable pain relief agents include, but are not limited to, aspirin, naproxen, ibuprofen, acetaminophen, oxycodone, hydrocodone, and morphine.

Examples of suitable analgesic agents include, but are not limited to, menthol, methyl salicylate, capsaicin, diclofenac, ketoprofen, and piroxicam.

Examples of suitable anesthetic agents include, but are not limited to, lidocaine, pramoxine, phenol, benzocaine, lidocaine, prilocaine, dibucaine, dyclonine, tetracaine, paroxine, pentafluoropropane, tetrafluoroethane, ethyl chloride, cocaine, and camphor.

Examples of suitable anti-inflammasome agents include, but are not limited to, direct NLRP3 inflammasome binders (e.g., MCC950, VX765, tranilast, parthenolide, disulfiram, inflazome, OLT1177), inhibitors of nucleotide-detecting receptors (e.g., A438079, apyrase, ox-ATP), and inhibitors of inflammasome pathway kinases (e.g., OXSI-2, fostamatinib, R406, BAY 61-3606).

Examples of suitable pro-healing bioactives include, but are not limited to, small molecule bioactives (e.g., histamine, insulin, ATP), growth factors (e.g., PDGFbb, SDF1, VEGF), and matrix components (e.g., collagen).

Examples of suitable antioxidants include, but are not limited to, retinoids, carotenoids, flavonoids, glutathione, selenium, ascorbic acid, green tea extract, resveratrol, pycnogenol, polypodium leucotomos, and astaxanthin.

In some aspects, the present disclosure provides compositions useful for inducing an immune response. In some embodiments, the composition comprises a vaccine and a polyvalent polymer. In some embodiments, the composition comprises a vaccine, and HA conjugated to a small molecule of Formula (I), HAA, and/or HAD.

Suitable vaccines include, but are not limited to, active whole-virus vaccines, attenuated whole-virus vaccines, inactivated whole-virus vaccines, active whole-bacteria vaccines, attenuated whole-bacteria vaccines, inactivated whole-bacteria vaccines, nucleic acid vaccines, protein vaccines, recombinant viral vaccines, nanoparticle vaccines, microparticle vaccines, and/or any combinations thereof.

### Wound Dressings

In some embodiments, the present disclosure provides functionalized wound dressings. In some embodiments, the functionalized wound dressings comprise a wound dressing saturated with, impregnated with, or embedded with a composition of the present disclosure or otherwise contain a composition of the present disclosure. In some embodiments, the functionalized wound dressing, upon contact with a wound, releases a polyvalent polymer from the composition of the disclosure into the wound.

In some embodiments, the wound dressing is in the form of a bandage, gauze, patch, pad, cloth, tape, film, or swatch. In some embodiments, the wound dressing comprises a means for affixing the wound dressing to tissue around the wound. Examples of suitable means include, but are not limited to, adhesives, pins, clasps, hooks, loops, ties, self-adhesive material, and the like. In some embodiments, the wound dressing and any means for affixing it are biodegradable.

In some embodiments, the wound dressing comprises a natural polymer or a synthetic polymer. Examples of suitable natural polymers for wound dressings include, but are not limited to, cellulose, starch, pectin, gelatin, chitosan, collagen, latex, alginate, carrageenan, silk fibroin, keratin, and derivatives thereof.

Examples of suitable synthetic polymers include, but are not limited to, polyethylene (PE), polysulfone (PS), polypropylene (PP), polylactic acid (PLA), poly(lactide-co-glycolide) (PLGA), polyglycoliyc acid (PGA), polycaprolactone (PCL), polyvinyl alcohol (PVA), polyvinyl chloride (PVC), polyurethane, polyvinylpyrrolidone (PVP), ethyl cellulose, methyl cellulose, nitrocellulose, poly(methacrylate-isobutene-monoisopropylmaleaste), polytetrafluoroethylene (PTFE), polyvinylidene fluoride (PVF), polyacrylate, polyamide, poly(ethylene oxide) (PEO), poly(glycolic-caprolactone), polyhydroxyalkanoate, polyanhydride, poly(orthocarbonate, polycarbonate, polyphosphonate, polysiloxanes, and functionalized and derivatized versions thereof.

In some embodiments, the dressing material may be configured as a hydrogel. A “hydrogel” refers to a semisolid composition constituting a substantial amount of water, in which polymers or mixtures thereof are dissolved or dispersed. The hydrogels may be physically or chemically crosslinked.

In some embodiments, the dressing material may include a crosslinker. For example, the crosslinker can be included with a natural polymer, semi-natural polymer, or synthetic polymer. The crosslinker can crosslink the material. “Crosslink” is defined as understood by those skilled in the art of polymer chemistry. In general, cross-linking refers to the formation of covalent bonds, or cross-links, between polymeric/macromolecular molecules. The crosslinking process also generally refers to a fixation process that stabilizes tissue by reducing its antigenicity and thus its susceptibility to enzymatic degradation. A “crosslinking agent” is defined as a compound capable of forming a crosslink. For example, glutaraldehyde is generally known in the art as a crosslinking agent for the tissue.

In some embodiments, the crosslinker can include poly(ethylene glycol) diglycidyl ether, glycerol diglycidyl ether, 1,4-butanediol diglycidyl ether, ethylene glycol glycidyl ether, polypropylene glycol diglycidyl ether, combinations thereof, or others.

### Methods

In some embodiments, the present disclosure provides a method of inducing cytokine release by a cell. In some embodiments, the method comprises inducing cytokine release by a macrophage. In some embodiments, the method comprises contacting a macrophage with a composition or functionalized wound dressing of the present disclosure.

In some embodiments, the method increases cytokine release by about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 220%, about 240%, about 260%, about 280%, about 300%, about 320%, about 340%, about 360%, about 380%, or about 400% relative to untreated or control treated (mock treatment, solvent, blank, etc.) cells.

In some embodiments, the method increases the release of at least one selected from the group consisting of IL-1β, TNF-α, and IL-6. In some embodiments, the method dose not increase the release of IL-4 and/or IL-10. In some embodiments, the method increases IL-1β release by about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 220%, about 240%, about 260%, about 280%, about 300%, about 320%, about 340%, about 360%, about 380%, or about 400% relative to untreated or control treated (mock treatment, solvent, blank, etc.) cells.

In some embodiments, the method increase TNF-α release by about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 220%, about 240%, about 260%, about 280%, about 300%, about 320%, about 340%, about 360%, about 380%, about 400%, about 420%, about 440%, about 460%, about 480%, about 500%, about 520%, about 540%, about 560%, about 580%, about 600%, about 620%, about 640%, about 660%, about 680%, or about 700% relative to untreated or control treated (mock treatment, solvent, blank, etc.) cells.

In some embodiments, the method increase IL-6 release by about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 220%, about 240%, about 260%, about 280%, about 300%, about 320%, about 340%, about 360%, about 380%, about 400%, about 420%, about 440%, about 460%, about 480%, about 500%, about 520%, about 540%, about 560%, about 580%, about 600%, about 620%, about 640%, about 660%, about 680%, about 700%, about 720%, about 740%, about 760%, about 780%, about 800%, about 820%, about 840%, about 860%, about 880%, or about 900% relative to untreated or control treated (mock treatment, solvent, blank, etc.) cells.

In some embodiments, the present disclosure provides a method of treating a wound in tissue. In some embodiments, the method comprises at least one selected from the group consisting of: increasing innate immune response in the wound area, increasing release of pro-inflammatory cytokines in the wound area, increasing intracellular calcium release, inducing keratinocyte migration in the wound, and inducing N2- and M2-polarized reparative cell recruitment to the wound.

In some embodiments, the method increases the innate immune response in a wound by increasing NF-κB activation and/or IR3 activation relative to an untreated wound. In some embodiments, the method increases NF-κB activation by about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 220%, about 240%, about 260%, about 280%, or about 300% relative to an untreated wound. In some embodiments, the method increases IRF3 activation by about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 220%, about 240%, about 260%, about 280%, or about 300% relative to an untreated wound.

In some embodiments, the method increases the release of pro-inflammatory cytokines from a cell. In some embodiments, the release of pro-inflammatory cytokines is increased by about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 220%, about 240%, about 260%, about 280%, about 300%, about 320%, about 340%, about 360%, about 380%, or about 400% relative to release from a cell in an untreated wound.

In some embodiments, the method increases the release of at least one selected from the group consisting of IL-10, TNF-α, and IL-6. In some embodiments, the method does not increase the release of IL-4 and/or IL-10. In some embodiments, the method increases IL-1β release by about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 220%, about 240%, about 260%, about 280%, about 300%, about 320%, about 340%, about 360%, about 380%, or about 400% relative to release from a cell in an untreated wound.

In some embodiments, the method increase TNF-α release by about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 220%, about 240%, about 260%, about 280%, about 300%, about 320%, about 340%, about 360%, about 380%, about 400%, about 420%, about 440%, about 460%, about 480%, about 500%, about 520%, about 540%, about 560%, about 580%, about 600%, about 620%, about 640%, about 660%, about 680%, or about 700% relative to release from a cell in an untreated wound.

In some embodiments, the method increase IL-6 release by about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 220%, about 240%, about 260%, about 280%, about 300%, about 320%, about 340%, about 360%, about 380%, about 400%, about 420%, about 440%, about 460%, about 480%, about 500%, about 520%, about 540%, about 560%, about 580%, about 600%, about 620%, about 640%, about 660%, about 680%, about 700%, about 720%, about 740%, about 760%, about 780%, about 800%, about 820%, about 840%, about 860%, about 880%, or about 900% relative to release from a cell in an untreated wound.

In some embodiments, the method increases intracellular calcium release in keratinocytes in a wound by about 10%, about 20%, about 30%, about 40%, about 50%, about 60%, about 70%, about 80%, about 90%, about 100%, about 110%, about 120%, about 130%, about 140%, or about 150% relative to an untreated wound.

In some embodiments, the method increases keratinocyte migration in a wound by about 1%, about 2%, about 3%, about 4%, about 5%, about 6%, about 7%, about 8%, about 9%, or about 10% relative to an untreated wound.

In some embodiments, the method increases recruitment of reparative cells to a wound. In some embodiments, the method increases recruitment of reparative cells to a wound by about 5%, about 10%, about 15%, about 20%, about 25%, about 30%, about 35%, about 40%, about 45%, about 50%, about 55%, about 60%, about 65%, about 70%, about 75%, or about 80% relative to an untreated wound.

In some embodiments, the method increases recruitment of N2- or M2-polarized reparative cells to a wound. In some embodiments, the method increases recruitment of N2-polarized reparative cells to a wound by about 5%, about 10%, about 15%, about 20%, about 25%, about 30%, about 35%, about 40%, about 45%, about 50%, about 55%, about 60%, about 65%, about 70%, about 75%, or about 80% relative to an untreated wound. In some embodiments, the method increases recruitment of M2-polarized reparative cells to a wound by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% relative to an untreated wound.

In some embodiments, a method of treating a wound in a tissue comprises applying a composition of the present disclosure to the wound and allowing the wound to heal in contact with the composition. In some embodiments, the method further comprises applying a wound dressing to the wound after applying the composition and allowing the wound to heal in contact with the previously applied composition.

In some embodiments, the method further comprises an additional step of removing the wound dressing and replacing it with a second wound dressing. In some embodiments, the method comprises readministering a composition of the present disclosure to the wound before replacing the wound dressing.

In some embodiments, the method comprises applying a functionalized wound dressing of the present disclosure to the wound and allowing the wound to heal in contact with a composition of the present disclosure contained within the functionalized wound dressing. In some embodiments, the method further comprises a step of removing the functionalized wound dressing and replacing it with a new functionalized wound dressing.

In some embodiments, the method comprises a step of rinsing or washing the wound before applying a composition or functionalized wound dressing to the wound.

Suitable solutions for rinsing and/or washing the wound include, but are not limited to, water, distilled water, saline, soap, ethanol, isopropanol, iodine, crystal violet, chlorhexidine, triclosan, tea tree oil, hydrogen peroxide, octenidine, polyhexanide, sodium hypochlorite, nanosilver, lactated physiologic saline solutions, buffered saline solutions, and combinations thereof.

In some embodiments, methods of the present disclosure comprise a step of fastening the wound dressing or functionalized wound dressing to the tissue. In some embodiments, the method comprises fastening the wound dressing or functionalized wound dressing using a fastening means that is part of the wound dressing or functionalized wound dressing. In some embodiments, the method comprises a step of fastening the wound dressing or functionalized wound dressing with an external fastening means. Examples of suitable fastening means include, but are not limited to, adhesives, pins, clasps, hooks, loops, ties, self-adhesive material, sutures, staples, clamps, and the like.

In some embodiments, the wound is an acute wound or a diabetic wound. In some embodiments, the wound is selected from a cut, tear, surface area wound, skin defect, or combination thereof.

In some embodiments, the wound has a size between about 1 mm and about 100 mm, about 2 mm and about 75 mm, about 3 mm and about 50 mm, about 4 mm and about 25 mm, or about 5 mm and about 10 mm.

In some aspects, the present disclosure provides methods of inducing an immune response. In some embodiments, the method comprises administering to a subject a composition comprising an immunogenic molecule, a virus, and/or an organism, and a polyvalent polymer. In some embodiments, the composition is a vaccine composition. In some embodiments, the composition is a vaccine composition comprising HA conjugated to a small molecule of the present disclosure, HAA, and/or HAD.

In some embodiments, administration of a vaccine composition of the present disclosure induces a greater immune response than administration of the same vaccine composition that lacks a polyvalent polymer.

In some embodiments, the method increases the innate immune response to the vaccine composition by increasing NF-κB activation and/or IR3 activation relative to administration of an identical vaccine composition lacking the polyvalent polymer. In some embodiments, the method increases NF-κB activation by about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 220%, about 240%, about 260%, about 280%, or about 300% relative to administration of an identical vaccine composition that lacks the polyvalent polymer. In some embodiments, the method increases IRF3 activation by about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 220%, about 240%, about 260%, about 280%, or about 300% relative to administration of an identical vaccine composition that lacks the polyvalent polymer.

### Pharmaceutical Compositions

In some aspects, the present disclosure provides compositions useful for treating wounds or inducing an immune response. In some embodiments, the composition comprises at least one polyvalent polymer. In some embodiments, the at least one polyvalent polymer comprises HA conjugated to a small molecule of Formula (I), HAA, and/or HAD. In some embodiments, the composition is a vaccine composition comprising an antigenic molecule and/or organism, and HA conjugated to a small molecule of Formula (I), HAA, and/or HAD.

In some embodiments, the composition of the present disclosure comprises a pharmaceutical composition. Formulations of the pharmaceutical compositions described herein may be prepared by any method known or hereafter developed in the art of pharmacology. In general, such preparatory methods include the step of bringing the active ingredient into association with a carrier or at least one other accessory ingredient, and, if necessary or desirable, then shaping or packaging the product into a desired single- or multi-dose unit.

Pharmaceutical compositions that are useful in the methods of the disclosure may be prepared, packaged, or sold in any formulations suitable for topical or internal administration.

A pharmaceutical composition of the disclosure may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage, such as, for example, one-half or one-third of such a dosage.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the disclosure may further comprise at least one additional pharmaceutically active agent. Other active agents include growth factors, hormones, anti-inflammatories, including corticosteroids, immunosuppressants, and the like.

Controlled- or sustained-release formulations of a pharmaceutical composition of the disclosure may be made using conventional technology.

Liquid preparations may be in the form of solutions, syrups, or suspensions. The liquid preparations may be prepared by conventional means with pharmaceutically acceptable additives such as suspending agents (e.g., methyl cellulose, hydrogenated fats); emulsifying agents (e.g., lecithin or acacia); non-aqueous vehicles (e.g., almond oil, oily esters, or ethyl alcohol); and preservatives (e.g., methyl or propyl p-hydroxy benzoates or sorbic acid). Liquid formulations of a pharmaceutical composition of the disclosure that are suitable for administration may be prepared, packaged, and sold either in liquid form or in the form of a dry product intended for reconstitution with water or another suitable vehicle before use.

Formulations of a pharmaceutical may comprise the active ingredient combined with a pharmaceutically acceptable carrier, such as sterile water or sterile isotonic saline, and include, but are not limited to, suspensions, solutions, emulsions in oily or aqueous vehicles, pastes, and implantable sustained-release or biodegradable formulations. Such formulations may further comprise at least one additional ingredient, including, but not limited to, suspending, stabilizing, or dispersing agents.

A pharmaceutical composition of the disclosure may be prepared, packaged, or sold in a formulation suitable for spray-on administration. Such compositions may be in the form of dry powders for administration using a device comprising a dry powder reservoir to which a stream of propellant may be directed to disperse the powder or using a self-propelling solvent/powder-dispensing container, such as a device comprising the active ingredient dissolved or suspended in a low-boiling propellant in a sealed container. Alternatively, the composition may be in solution form, in which droplets or foam are propelled by the release of a low-boiling propellant in a sealed or manually pressurized container.

Low-boiling propellants generally include liquid propellants with a boiling point below 65° F. at atmospheric pressure. By way of example, the propellant may constitute 50 to 99.9% (w/w) of the composition, and the active ingredient may constitute 0.1 to 20% (w/w) of the composition. The propellant may further comprise additional ingredients, such as a liquid non-ionic or solid anionic surfactant, or a solid diluent (preferably having a particle size of the same order as the particles comprising the active ingredient).

As used herein, “additional ingredients” include, but are not limited to, at least one of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the disclosure are known in the art and described, for example, in Remington's Pharmaceutical Sciences (1985, Genaro, ed., Mack Publishing Co., Easton, PA), which is incorporated herein by reference.

### EXAMPLES

### Example 1: Synthesis and Investigation of Hyaluronic Acid-Derived Biomaterials

Currently, biopolymers with good biocompatibility and degradability, including polysaccharides, have been widely studied for wound healing. However, some of these polymers have drawbacks, including solubility, microbial contamination, toxicity, and efficacy in drug delivery. To overcome some of these challenges, biomaterials that are safe, non-toxic, and have good stability and wound-closure properties were designed and developed, with histamine or histidine conjugated to low-, medium-, and high-molecular-weight hyaluronic acid.

Histamine, as one of the mediators released from mast cells, has been widely studied for its role in wound healing. It was suggested that exogenous histamine promoted angiogenesis and macrophage recruitment by upregulating bFGF levels in wounds. Histamine stimulated fibroblast proliferation, production of αSMA, and collagen synthesis (Numata, Y., et al., 2006, Journal of Investigative Dermatology, 126(6):1403-1409; Lohi, J., et al., 1992, Journal of Cellular Biochemistry, 50(4):337-349; Ghosh, D., et al., 2024, Biomaterials, 306:122496). It was noted that L-histidine and 1-carnosine improve proliferation, migration, senescence, and epithelial-mesenchymal transition (EMT) to accelerate wound healing in vitro and in vivo (Kim, Y., et al., 2020, Nutritional Research and Practice, 14(3):188-202). In another study, histidine promoted astrocyte migration mediated by the H2 receptor towards the infarct core to support long-term neurological recovery after cerebral ischemia (Liao, R. J., et al., 2015, Scientific Reports, 5:15356).

Hyaluronic acid (HA) is a natural linear glycosaminoglycan polysaccharide, consisting of repeating units of (1-4)-d-glucuronic acid and (1-3)-N-acetyl-d-glucosamine. High-molecular-weight HA (>100 kDa) is a major constituent of the extracellular matrix (ECM) in the human body, present in most skin tissues (dermis and epidermis), brain and central nervous system (Dicker, K. T., et al., 2014, Acta Biomaterials, 10(4):1558-1570; Iaconisi, G., N., et al., 2023, International Journal of Molecular Sciences, 24(12):10296; Snetkov, P., et al., 2020, Polymers, 12(8):1800; Frenkel, J. S., 2014, International Wound Journal, 11(2):159-163; Antoszewska, M., et al., 2024, Scientia Pharmaceutica, 92(2):23). During early stages of wound healing process, HA plays an important role in supporting cells in facilitating transport of nutrients and waste products and activates keratinocyte migration and proliferation (De Francesco, F., et al., 2023, European Review for Medical and Pharmacological Sciences, 27(3):37-45; Yang, H., 2021, ACS Applied Bio Materials, 4(1):311-324; Della Sala, F., et al., 2022, Applied Sciences, 12(6):3091). HA is actively degraded by the hyaluronidase enzymes or by reactive oxygen species, which plays an important role in collagen deposition and angiogenesis and plays a key role in skin homeostasis during healing process (Aya, K. L., et al., 2014, 22(5):579-593; Ding, Y. W., et al., 2022, Biomaterial Sciences, 10(13):3393-3409; Raza, H., et al., 2023, Scientific Reports, 13(1):13425). HA, due to its scavenging activity, helps in protecting granulation tissue from oxygen free radicals (Trabucchi, E., et al., 2002, International Journal of Tissue Reactions, 24(2):65-71; Valachova, K., et al., 2021, International Journal of Molecular Sciences, 22(13):7077; Gao, F., et al., 2010, Matrix Biology, 29(2):107-116). Hyaluronic acid, due to its biocompatibility and non-antigenic profile, has been investigated in the efficacy of chronic wounds and in several clinical applications, including arthritis, and accelerating and improving the healing of chronic wounds (Voinchet, V., et al. 2006, American Journal of Clinical Dermatology, 7(6):353-357; Zhang, X., et al., 8:516; Tokatlian, T., et al., 2015, Advanced Healthcare Materials, 4(7):1084-1091; Marinho, A., et al., 2021, Biomolecules, 11(10):1518; Nyman, E., et al., 2019, Plastic and Reconstructive Surgery—Global Open, 7(5):e2221). Exogenous HA was found to accelerate skin wound healing in mice, rats, hamsters, and diabetic foot ulcers (Takahashi, Y., et al., 2005, Journal of Biological Chemistry, 280(25):24195-24204; Rajapaksa, S., P., et al., 2005, American Journal of Rhinology & Allergy, 19(6):572-576; Schimizzi, A. L., et al., 2006, Spine Journal, 6(5):550-556; Proctor, M., et al., 2006, American Journal of Rhinology & Allergy, 20(2):206-211).

### Synthesis and Characterization of the Histamine and Histidine Conjugated Hyaluronic Acid Polymers.

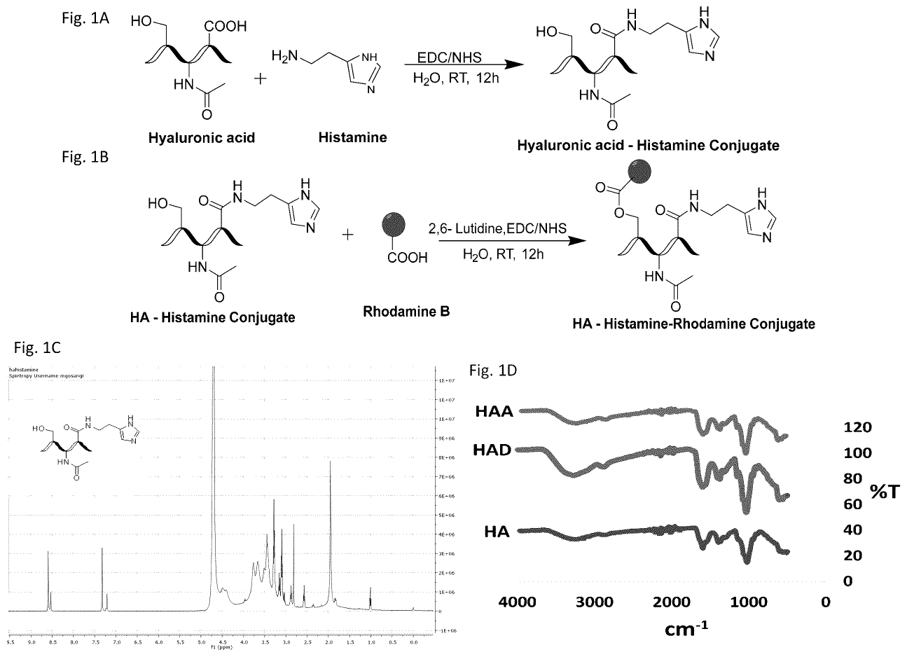

The synthesis of the hyaluronic acid (low—LMW, medium—MMW, and high-molecular-weight—HMW) conjugation with histamine (hyaluronic acid-histamine—HAA) or histidine (hyaluronic acid-histidine—HAD) was carried out in aqueous phase at room temperature, as shown in FIG. 1. The carboxylic acid groups of hyaluronic acid polymer were coupled with the amino groups of the histamine or histidine in the presence of 1-ethyl-3-[3-dimethylaminopropyl]carbodiimide (EDC) and N-hydroxy succinimide (NHS), resulting in the formation of the hyaluronic acid-histamine (HAA) or hyaluronic acid-histidine (HAD) conjugates.

A ¹H NMR spectrum of the HAA is shown in FIG. 1. The chemical shifts of hyaluronic acid protons corresponding to the glucuronic acid group and the N-acetylglucosamine group are at 6=3.3 to 5.2 ppm and 6=2.0 to 4.5 ppm, respectively.

The chemical shifts corresponding to —CH₃—CO—NH protons of the N-acetylglucosamine group appeared at δ=2.0-2.2 ppm. The chemical shifts of the protons corresponding to the imidazole ring at δ=7.4 to 8.65 ppm indicate the formation of HAA and HAD polymers. The chemical shifts corresponding to the protons of the ethylamine of histamine or histidine (δ=3.1 to 3.3 ppm) also confirm the formation of HAA or HAD polymers. Downfield shifts were also observed in the 1H NMR spectra of histamine- or histidine-conjugated hyaluronic acid polymers compared to free hyaluronic acid, histamine, or histidine. The degree of conjugation of histamine or histidine was also calculated using ¹H NMR spectra to be ~28-38% in all HA-conjugated polymers (Table 1).

TABLE 1
Polymer Derivatization

| Polymer | % Derivatization |
|---|---|
| HAA-LMW | 34.9 ± 4.5 |
| HAD-LMW | 37.5 ± 3.3 |
| HAA-MMW | 31.4 ± 3.6 |
| HAD-MMW | 29.3 ± 4.5 |
| HAA-HMW | 28.6 ± 3.1 |
| HAD-HMW | 30.7 ± 2.9 |

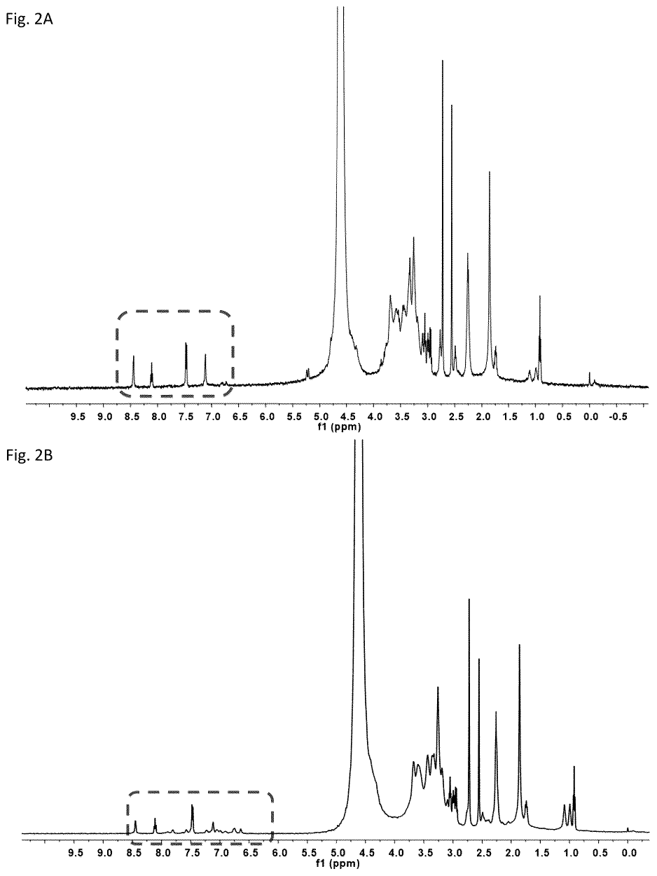

¹H NMR spectra of rhodamine conjugated hyaluronic acid polymer (HAR), rhodamine conjugated to HAA or HAD polymers, were also investigated in D₂O. The chemical shifts corresponding to the protons of rhodamine at δ=7.0 to 8.5 ppm indicate the formation of HAR, HAAR, and HADR polymers (FIG. 2).

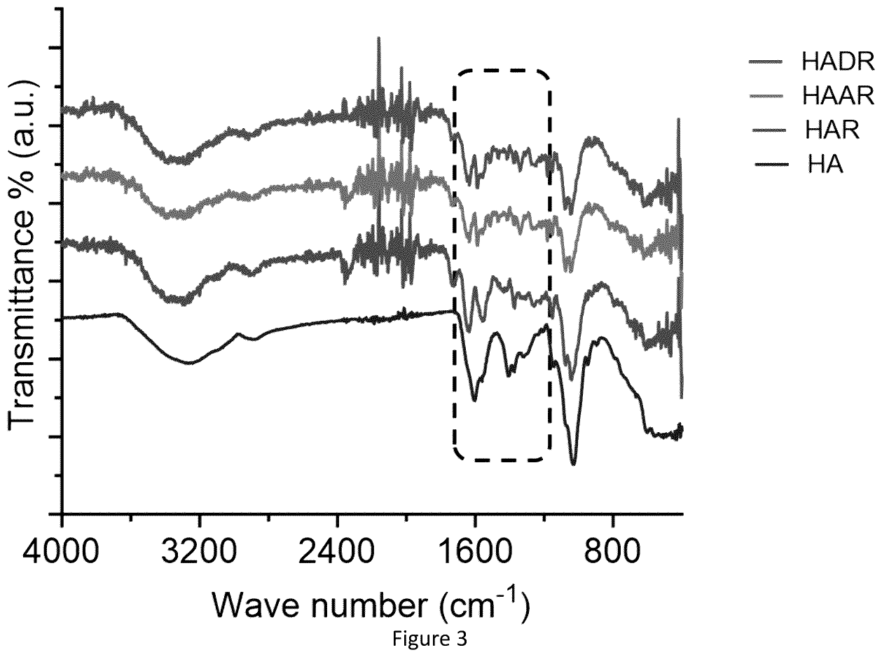

The conjugation of histamine or histidine to hyaluronic acid polymer was further confirmed using FT-IR spectroscopy. As shown in FIG. 1, the band at 3436 cm⁻¹ corresponds to N—H and O—H stretching vibrations, while the bands at 2876 cm⁻¹ and 1603 cm⁻¹ correspond to C—H and N—H stretching vibrations, respectively. Significant changes were found in the spectrum of histamine or histidine-conjugated HA polymers compared to that of the parental HA polymer. The peaks at 1600 cm⁻¹ and 3100 cm⁻¹ in the HAD polymer in FIG. 1 can be attributed to OC—NH stretching vibration (amide bond formation), indicating conjugation of histamine or histidine to hyaluronic acid. The ester peaks at 1550 cm⁻¹ correspond to C—O stretching vibrations, confirming the rhodamine conjugation to HAA and HAD polymers (FIG. 3).

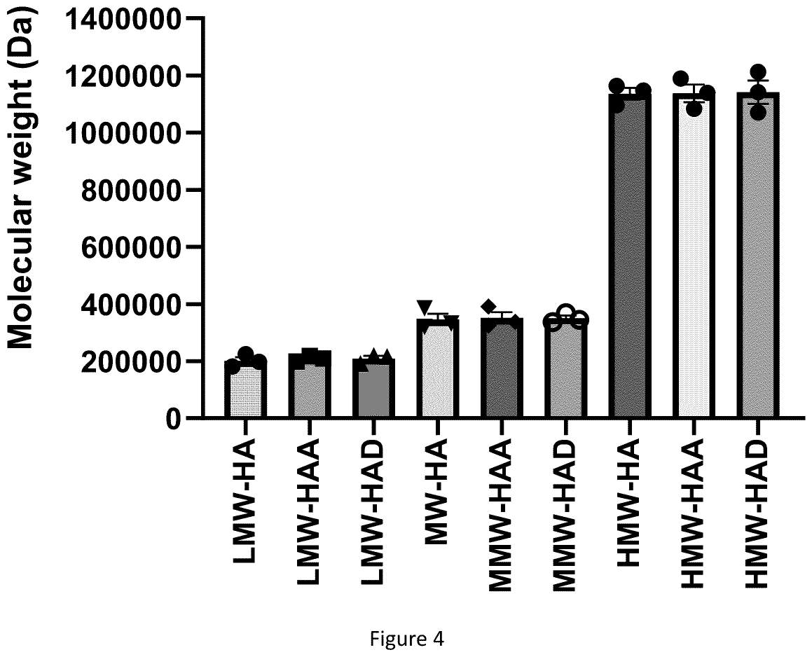

Gel permeation chromatography (GPC) was employed to determine the molecular weights of all hyaluronic acid conjugates (low-, medium-, and high-molecular weights) (FIG. 4). The conjugated polymers showed slightly higher molecular weights, resulting from small-molecule conjugation to the acid groups of hyaluronic acid monomers (L-histidine and histamine dihydrochloride; molecular weights: 228.08 and 115.15 g/mol, respectively).

The hydrodynamic diameters and zeta potentials of all HA polymers were determined to understand their particle formation behavior (Table 2). Hydrodynamic diameters ranging from 750 nm to 1550 nm were observed, and low molecular weight HA conjugates showed slightly lower hydrodynamic diameters than high-molecular-weight HA conjugates.

TABLE 2
Dynamic Light Scattering (DLS) of Polymers

| | Polydispersity | Hydrodynamic | Zeta Potential |
|---|---|---|---|
| Polymer | Index (PDI) | Diameter (nm) | (mV) |
| HA-LMW | 0.892 ± 0.45 | 829 ± 82.8 | −12.23 ± 3.3 |
| HAA-LMW | 0.958 ± 0.58 | 738 ± 68.9 | −8.34 ± 5.2 |
| HAD-LMW | 0.535 ± 0.36 | 847 ± 40.2 | −10.90 ± 4.6 |
| HA-MMW | 0.988 ± 0.62 | 1133 ± 71.8 | −14.60 ± 8.2 |
| HAA-MMW | 0.874 ± 0.59 | 1237 ± 67.4 | −11.28 ± 7.5 |
| HAD-MMW | 0.785 ± 0.64 | 1538 ± 109.2 | −12.80 ± 6.3 |
| HA-HMW | 0.960 ± 0.48 | 1427 ± 81.5 | −15.25 ± 9.7 |
| HAA-HMW | 0.758 ± 0.56 | 1432 ± 73.1 | −12.20 ± 6.2 |
| HAD-HMW | 0.955 ± 0.70 | 1356 ± 82.8 | −12.92 ± 5.6 |

This is likely due to the polymer-hindered structure and the absence of lipid amphiphiles, which may lead to smaller hydrodynamic diameters. Intermolecular hydrogen bonding between hydroxyl groups and free acid molecules may also contribute to the formation of complex structures and to increased hydrodynamic diameters. A reduction in the hydrodynamic diameters of low-molecular-weight polymer conjugates (approximately 740-850 nm) was observed. Zeta potential values of HA polymers ranged from −0.2 to −30 mV, and typically increased with increasing amount of histamine or histidine employed in the polymers (Table 1).

In Vitro Innate Immune Responses Induced with Hyaluronic Acid Polymers:

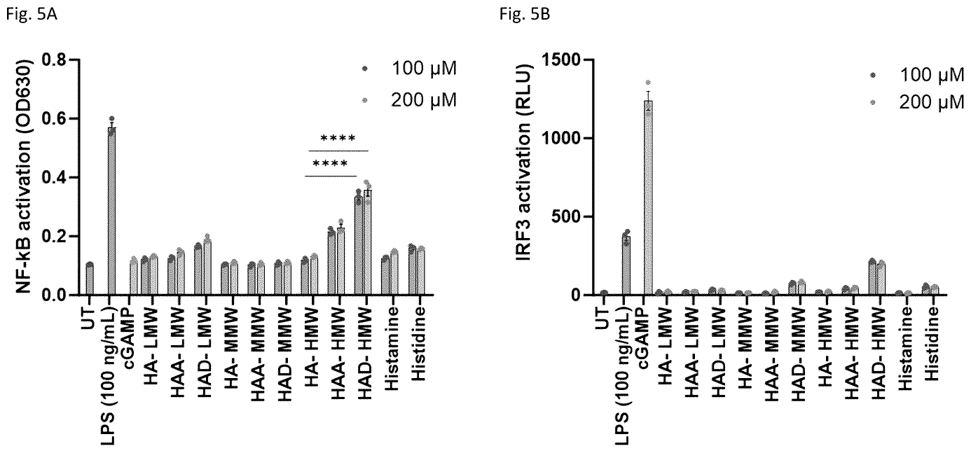

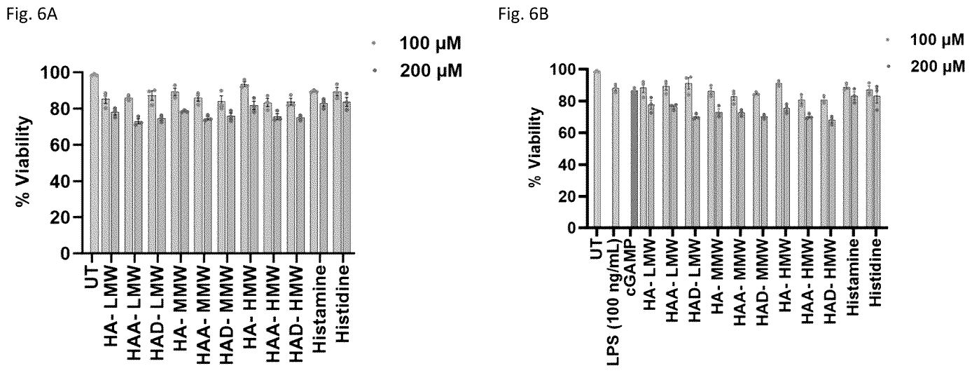

The innate immune response of HA polymer derivatives was investigated in J774-Dual macrophages using a reporter protein assay for the NF-κB pathway and IRF3. J774-Dual cells were seeded at a density of 100,000 cells/well in 96-well plates and treated with DMEM (negative control), LPS (100 ng/mL, NF-κB positive control), cGAMP (100 μM, IRF3 positive control), or HA polymers (free HA, HAA, and HAD, 100 μM and 200 μM). Histamine and histidine (100 μM) were also used as internal controls. Significant enhancement of NF-κB activation was observed with both concentrations (100 μM and 200 μM) of HAD-HMW treatments compared to all other treatments. However, no significant difference was observed between 100 M and 200 M treatments. The activation of macrophages was indicative of the innate immune response to HMW-HAD polymers. The results also showed increased IRF3 activation with HMW-HAD polymer treatment (FIG. 5). The polymers were also tested for toxicity at the same concentrations, and the results indicate that they are minimally toxic to macrophages and keratinocytes (FIG. 6).

In Vitro Cytokine Release with Hyaluronic Acid Polymers:

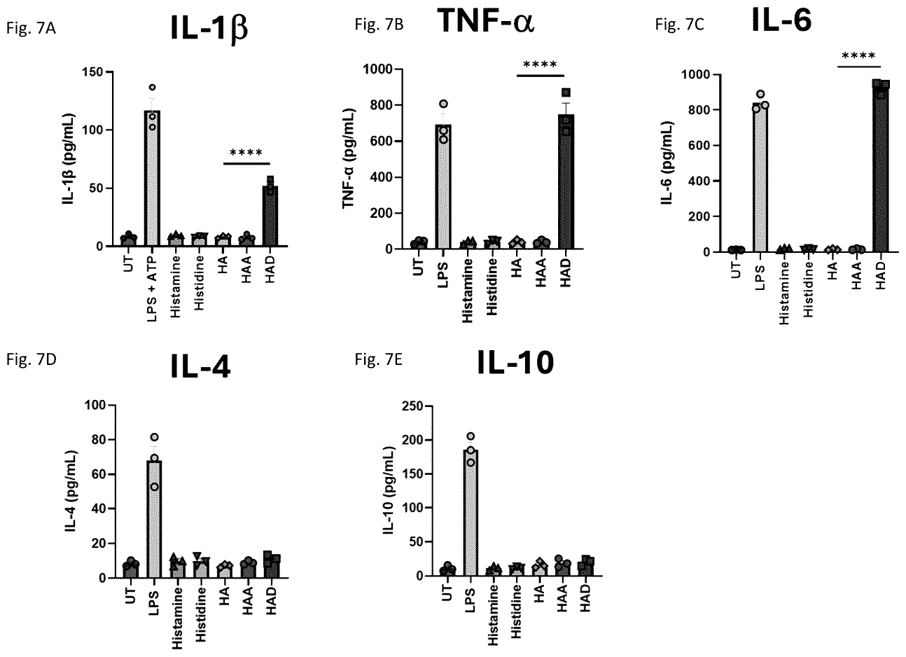

The inflammatory response from J774-Dual macrophages was measured as production of three inflammation mediators, IL-1β, TNF-α, and IL-6. J774-Dual macrophages were stimulated by LPS (0.1 g/mL) or with HA polymers, and ELISA analysis was performed. These cytokine productions are reported in FIG. 7. The results obtained indicate that, after LPS stimulation or treatment with HAD-HMW, J774-Dual macrophages release significant amounts of IL-1β, TNF-α, and IL-6. While IL-1β release was higher in LPS-stimulated cells, TNF-α and IL-6 levels were higher in cells treated with HAD polymer. By contrast, when macrophages were treated with histamine, histidine, or other polymers, no significant production of cytokines was observed (FIG. 7), indicating that HAD polymer has inflammatory properties, which are important effects at early stages of wound healing, stimulating the proliferation and chemotaxis of fibroblasts and increasing the production of extracellular matrix proteins. Of interest is observation that HAD-HMW does not drive release of IL-4 and IL-10, which LPS does (FIG. 7D and FIG. 7E). These results highlighted and confirmed an important role of the HAD polymer in modulating the inflammatory response, extending its potential application to diabetic ulcers and chronic wounds.

### Rhodamine Conjugated Hyaluronic Acid Polymer Uptake Studies:

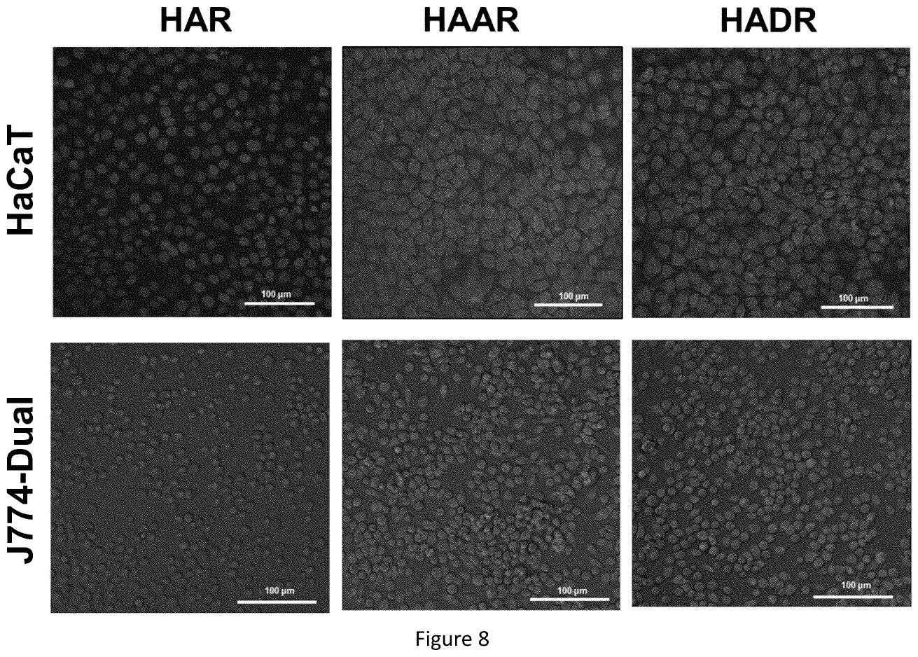

The cellular uptake and intracellular distribution of rhodamine conjugated to high-molecular-weight hyaluronic acid and its histamine and histidine conjugates in HaCaT cells (keratinocytes) and J774-Dual (macrophages) were visualized using confocal fluorescence microscopy. 50,000 cells were seeded in 8-chamber ibidi slides, incubated overnight, and treated with 100 μM rhodamine-conjugated polymers. After 24 hours of incubation, strong red fluorescence emission of rhodamine was observed mainly in the cytoplasm of cells treated with HAAR and HADR (FIG. 8). However, no rhodamine fluorescence was observed in the cytoplasm of cells treated with hyaluronic acid-rhodamine conjugate (HAR). These observations indicated that either histamine or histidine conjugation to hyaluronic acid induces entry of polymers into the cytoplasm, likely via endocytosis. The uptake of these polymers was observed to differ between keratinocytes and macrophages, with HAAR more internalized in the cytoplasm of keratinocytes and HADR in the cytoplasm of macrophages. The overall intensity of the red fluorescence observed in the cytoplasm of keratinocytes was greater than that observed for macrophages, indicating higher cellular entry of the polymers into keratinocytes. The results showed that the rhodamine-conjugated histamine or histidine polymers HAAR and HADR were internalized efficiently and transported to the cytoplasm.

### Calcium Ion Signaling in Keratinocytes:

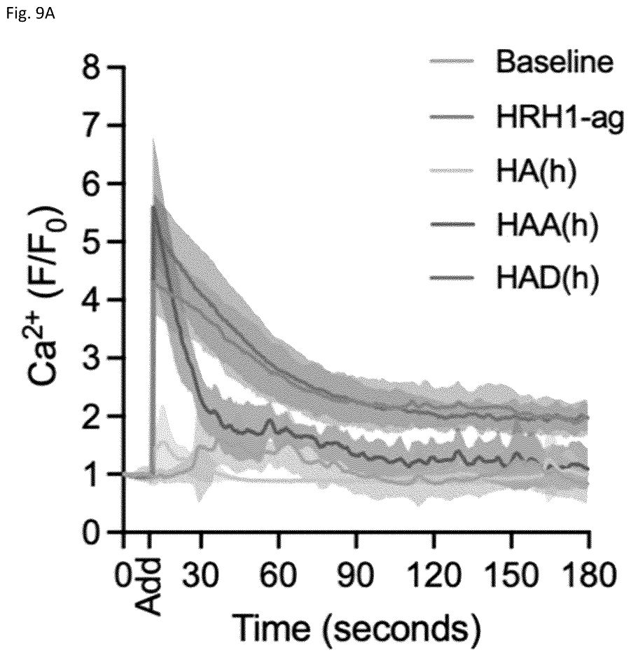

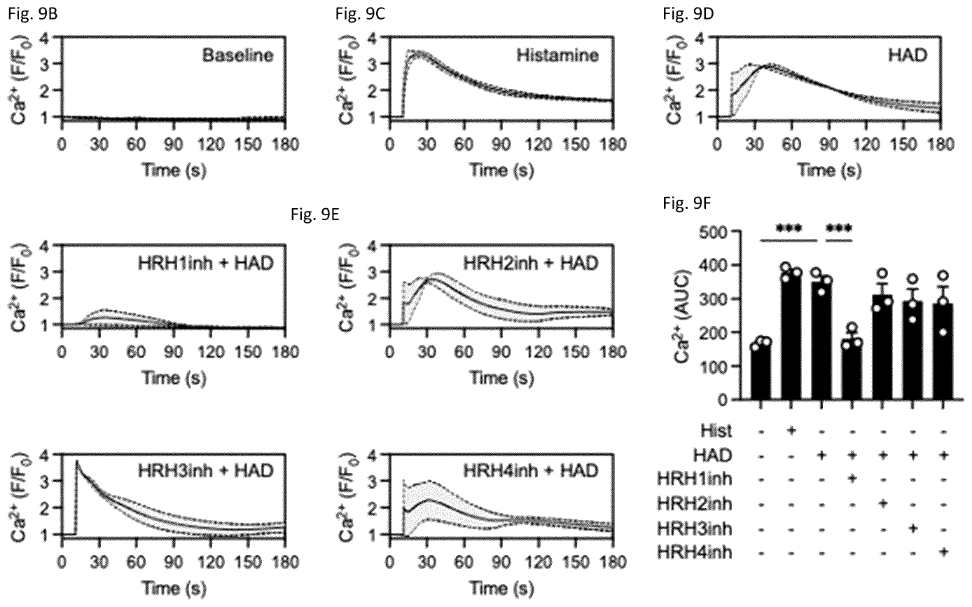

Intracellular Ca²⁺ ion signal transduction was investigated using keratinocytes treated with either histamine, HA polymers, or the HRH1 agonist 2-pyridylethylamine (positive control). It was observed that cells treated with the HA polymer did not exhibit Ca2+ signaling, whereas HAA or HAD treatments induced Ca2+ signaling in HaCaT cells, indicating that histamine or histidine conjugation results in intracellular Ca2+ flux. To check the specific histamine receptor-mediated Ca2+ ion signal transduction pathway, HaCaT cells were pretreated with histamine receptor antagonists (HRH1-4) for 30 minutes, HAD polymer was added, and the Ca2+ ion signal was measured. In FIG. 9, the results demonstrated that the Ca2+ ion signal was significantly inhibited by HRH1 inhibitor pretreatment, indicating that intracellular Ca2+ ion signaling in keratinocytes with conjugated HA polymers follows a histamine receptor 1-mediated Ca2+ ion signaling pathway.

In Vitro Keratinocyte Migration with Hyaluronic Acid Polymers:

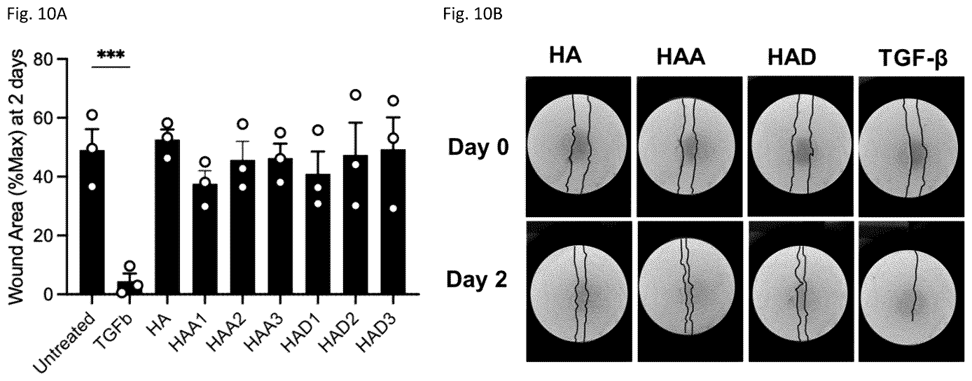

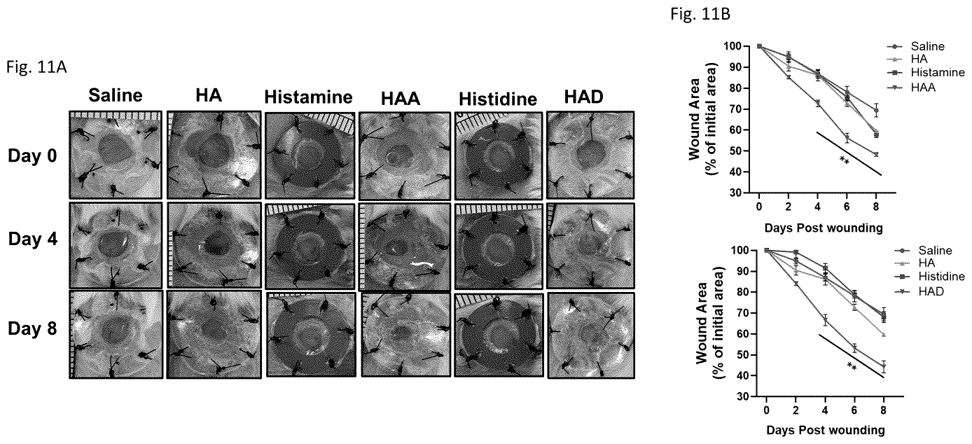

HaCaT cells were grown to confluence as a monolayer in a 24-well plate and scratched with a sterile 200 μL pipette tip. The cells were washed twice with PBS, treated with 1 ng/mL TGF-β, or 100 M HA, HAA, or HAD, or left untreated, and incubated at 37° C. with DMEM containing 1% FBS. Cells were observed 24 and 48 hours post-scratching to assess the effect on scratch closure rate by keratinocyte migration. The distance between the edges of the wound monolayer was measured in ImageJ as the mean distance. The quantified scratch-closure results for three batches of HA polymer, freshly prepared under three different conditions, at 48 hours, are shown in FIG. 10. After 48 hours, the wound closure in the control samples was less than 50%, whereas HA polymer treatments showed 55-58% closure. At 48 hours, the scratch was almost closed in the presence of the TGF-β (96-98%), while untreated cells (UT) and HA were all less than 55%. These observations indicate that there was no statistical difference among histamine- or histidine-conjugated hyaluronic acid biomaterials in their ability to induce keratinocyte proliferation. These results are likely due to the absence of immune cells (neutrophils, macrophages) and blood vessels in vitro, which support cell proliferation and migration in vivo.

### Efficacy of Conjugated Hyaluronic Acid Polymers in In Vivo Wound Healing:

The effects of HA-HMW, HAA-HMW, and HAD-HMW on the treatment of splinted full-thickness wounds were also examined in BALB/c mice (FIG. 11). A single dose of HA, HAA, or HAD (30 mg/kg) was prepared in 20 μL of saline and applied topically to the wound bed. Planimetric measurements were performed daily for 8 days, and normalized wound areas were calculated to track wound closure over time. The saline-treated group exhibited ~35% wound closure 8 days post-wounding, whereas HA-HMW treatment resulted in ~40% wound closure. HAA and HAD were observed to further accelerate wound closure, resulting in ~55% and ~57% wound closure, respectively (FIG. 11B). The results demonstrate that after only four days, HAA-HMW and HAD-HMW treatment of wounds resulted in >50% wound closure, indicating accelerated closure (FIG. 11A), while saline and HA-HMW treatment resulted in <40% wound closure over eight days. This clearly shows that HAA and HAD polymers improve wound closure kinetics from day 4 onwards compared with saline or HA delivery after wounding.

### Histology and Immunohistochemistry:

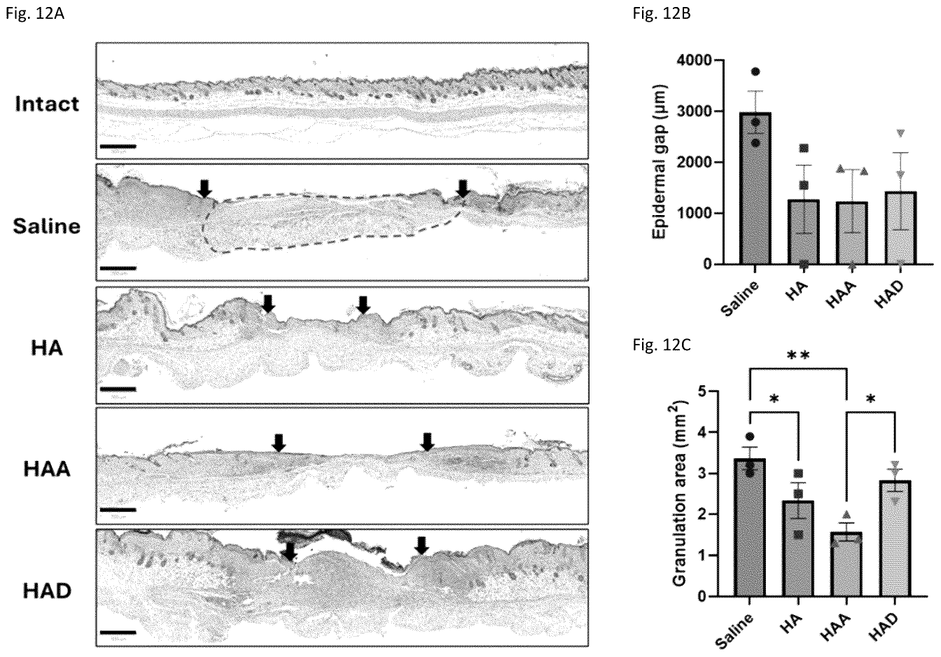

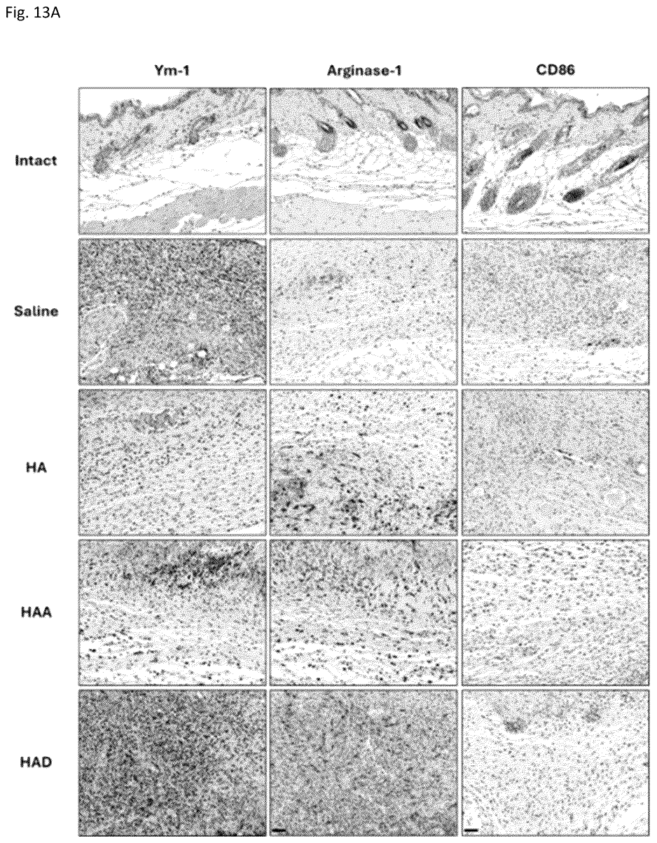

To study tissue morphology and cellular infiltration in the wound, tissue sections were stained with hematoxylin and eosin. Digital photographs of the stained tissues for all groups are shown in FIG. 12. While wounds in 1 out of 3 mice in the HA, HAA, and HAD groups had completely re-epithelialized, quantification of the epidermal gap (FIG. 12B) suggested that the differences between the treatment groups were not statistically significant. Next, granulation tissue deposition in the wound bed was quantified, indicating that the treatments did not significantly affect this process (FIG. 12C).

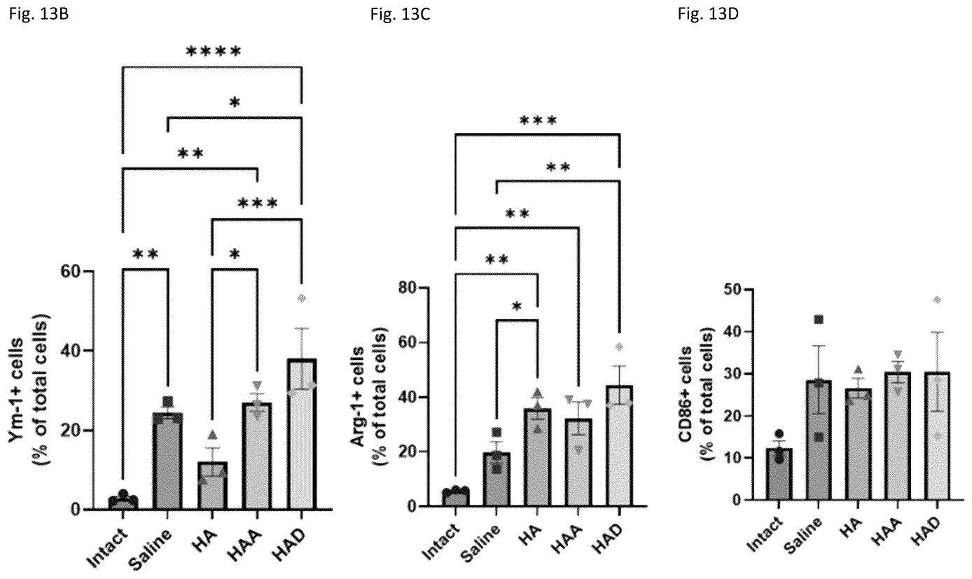

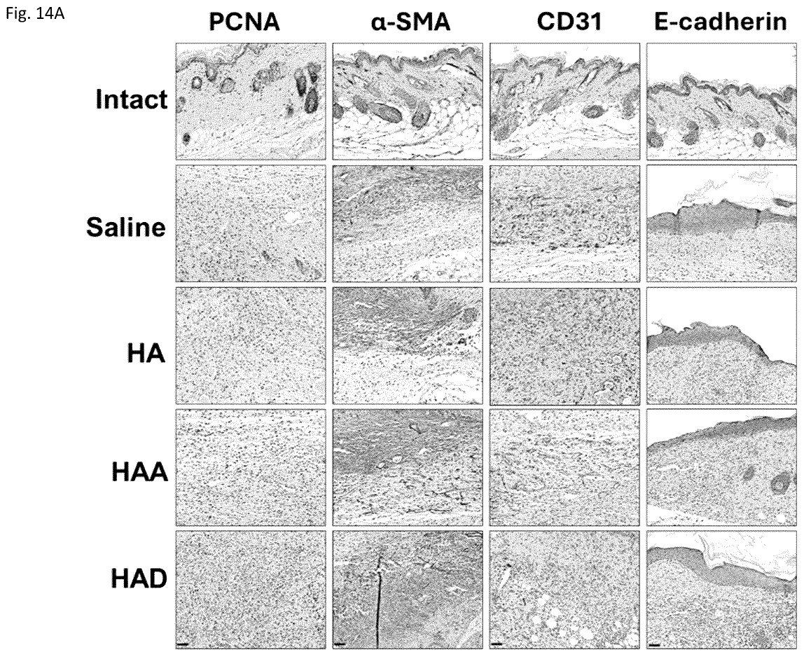

Immunohistochemical analyses were performed on skin harvested 8 days post-wounding to study the healing phenotype and progression. The proportions of different immune cell types and their polarization were quantified to assess the recruitment of innate immune cells (neutrophils and macrophages) to the wound microenvironment. FIG. 13 shows images of stained tissue, illustrating the density of positive detections (in brown) within a given wound area. Treatment of wounds with HAD polymer attracted significantly higher numbers of pro-reparative N2-polarized neutrophils compared to saline (FIG. 13A and FIG. 13B). Additionally, conjugation of histamine or histidine was found to significantly increase the number of N2 neutrophils compared to parental HA polymer (FIG. 13B). Pro-reparative M2-polarized macrophages were also found to be present in significant numbers in wounds treated with HA and HAD compared to saline (FIG. 13C). It has previously been demonstrated that high-molecular-weight hyaluronic acid promotes activation of pro-reparative macrophages with increased arginase 1 activity (Rayahin, J. E., et al., 2015, ACS Biomaterials Science & Engineering, 1(7):481-493). The number of pro-inflammatory CD86⁺ cells was comparable across all treatments (FIG. 13D). Overall, it can be concluded that histidine conjugation to HA promoted a pro-regenerative immune response in the wound.

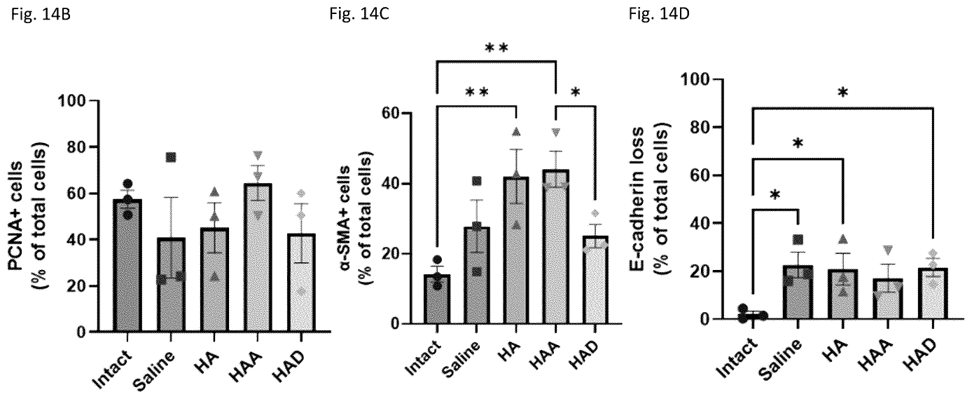

The progression of healing cell proliferation was comparable across groups, as measured by proliferating cell nuclear antigen (PCNA) expression, with proliferating cells observed in all skin layers (FIG. 14). Notably, wounds that progressed further in healing and had completed re-epithelialization showed a higher proportion of proliferating basal keratinocytes and other cells in the wound bed (FIG. 14B). Activated fibroblasts, characterized by α-smooth muscle actin (α-SMA) expression, primarily occupied the wound edge, with minimal presence in the wound bed. The number of these cells (also called myofibroblasts) was comparable among saline-, HA-, and HAA-treated wounds (FIG. 14C); however, their numbers were significantly lower in HAD-treated wounds than in HAA-treated wounds, suggesting a distinct interaction between histidine and histamine and cells (FIG. 14C). Loss of E-cadherin in keratinocytes, a marker of epithelial-to-mesenchymal transition, was also comparable across groups (FIG. 14D).

The materials and methods utilized are described herein.

### Materials:

High-molecular-weight hyaluronic acid (Mw=1000-2000 kDa) and low- and medium-molecular-weight hyaluronic acid (M_{w}=150-300 kDa) were purchased from Matexcel, USA. 1-ethyl-3-(3-dimethylaminopropyl) carbodiimide (EDC), N-hydroxy succinimide (NHS), 2,6-lutidine, L-histidine dihydrochloride, histamine dihydrochloride, and rhodamine B were purchased from Sigma-Aldrich. ELISA detection kits for IL-1β, TNF-α, and IL-6 were purchased from RD Systems (DuoSet), USA. A 3-(4,5-dimethylthiazol-2-yl)-2,5-diphenyltetrazolium bromide (MTT) assay kit was purchased from ATCC. Dulbecco's modified Eagle's medium (DMEM), fetal bovine serum (FBS), and trypsin-EDTA were purchased from Invitrogen. All reagents and solvents were used as received without further purification unless otherwise mentioned.

### Synthesis of Hyaluronic Acid-Derived Polymers:

The synthetic procedures using general EDC/NHS coupling reagents have been described previously (Miryala, B., et al., 2016, Colloids and Surfaces B: Biointerfaces, 146:924-937). Low-, medium-, or high-molecular-weight hyaluronic acid (1 eq, 0.0003 mmol) was dissolved in 20 mL of Nanopure™ water. EDC (3 eq, 0.001 mmol) and NHS (3 eq, 0.001 mmol) were added, and the reaction mixture was stirred for 20 minutes at room temperature. Histamine (3 eq, 0.001 mmol) or histidine (3 eq, 0.001 mmol) was added, and the reaction was stirred at room temperature for 24 hours. The resulting product was dialyzed against Nanopure™ water using a 3,500 Da molecular weight cut off (MWCO) for 3 days, with the water changed twice daily. The dialyzed product was then lyophilized to obtain the histamine-conjugated hyaluronic acid (HAA) or histidine-conjugated hyaluronic acid (HAD) polymer.

To obtain rhodamine conjugated polymers, rhodamine B (3 eq, 0.001 mmol) was dissolved in 15 mL of Nanopure™ water. 20 μL of 2,6-lutidine (mild base) was added, and the solution was stirred for 30 minutes. EDC (3 eq, 0.001 mmol) and NHS (3 eq, 0.001 mmol) were added, and the reaction mixture was stirred for 30 minutes at room temperature. The hyaluronic acid polymers HA, HAA, or HAD (1 eq, 0.0003 mmol) were added, and the reaction was stirred at room temperature for 24 hours. The products were centrifuged at 3,000 rpm for 15 minutes, and the supernatant was dialyzed against Nanopure™ water using a 3,500 Da molecular weight cutoff (MWCO) for 3 days, with the water changed every 6 hours. The dialyzed product was then lyophilized to obtain the rhodamine-conjugated HA (HAR), HAA (HAAR), or HAD (HADR) polymer.

### Characterization of Polymers:

The hyaluronic acid-conjugated polymers HAA, HAD, HAAR, and HADR were characterized using ¹H NMR, FT-IR, and GPC before use in further studies. The following characterization studies were carried out.

### Structural Characterization:

Structural compositions of HAA, HAD, HAAR, and HADR polymers were analyzed by 1H NMR (Varian 400 MHz instrument) in D2O, with tetramethylsilane (TMS) as the internal reference. The molecular weights of the polymers were analyzed using gel permeation chromatography (GPC; Waters Corporation, MA). Infrared (IR) spectra were determined using FT-IR spectroscopy with a PerkinElmer Frontier FTIR with a Pike Diamond ATR instrument to investigate the conjugation of histamine and histidine to hyaluronic acid.

### Hydrodynamic Diameter and Zeta Potential Values of HAA and HAD Polymers:

Hydrodynamic diameters and zeta potentials of HAA and HAD polymers in phosphate-buffered saline (1 mg/mL, 1×PBS, pH 7.4) were determined using a Zetasizer Nanosystems Nano-ZS instrument (Malvern Instruments, USA).

### Cell Culture:

HaCaT cells (human keratinocytes) and J774-Dual macrophages were cultured in DMEM supplemented with 10% FBS and 1% penicillin/streptomycin solution in an incubator containing 5% CO₂ at 37° C. At approximately 80% confluence, cells were scratched with a cell scraper (macrophages) or trypsinized, seeded at a density of 100,000 cells/well in 96-well plates (Corning, NY, USA), and allowed to attach overnight for subsequent experimentation.

### Cell Viability of HA Polymers:

HaCaT cells and J774-Dual macrophages were seeded in a 96-well plate at a density of 50,000 HaCaT cells/well and 100,000 J774-Dual macrophages/well in 200 μL DMEM medium with 10% FBS and antibiotics. Cells were incubated at 37° C. overnight, treated with HA, HAA, or HAD in 1×PBS, and then incubated for an additional 24 hours. Untreated control wells were treated only with fresh DMEM. After 24 hours, 10 μL of MTT ((3-(4,5-Dimethylthiazol-2-yl)-2,5-diphenyltetrazolium bromide) reagent was added, and cells were incubated for 3 hours at 37° C. Then, 50 μL of dimethyl sulfoxide and methanol (1:1 v/v) was added. Cells were incubated at room temperature for 10 minutes. The MTT metabolic assay was employed as a reporter of cell viability (Godeshala, S., et al., 2020, Journal of Materials Chemistry B, 8(37):8558-8572). After incubation, the contents of the wells were thoroughly mixed, and absorbance was determined at 570 nm using a plate reader (Bio-Tek Synergy 2).

Inflammation Response from Macrophages with HA Polymer Treatment:

The activation of innate immune response pathways induced by all HA polymer derivatives was investigated in J774-Dual macrophages in vitro. J774-Dual cells seeded at a density of 100,000 cells/well in 96-well plates to simultaneously study the NF-κB pathway, by assessing the activity of a secreted embryonic alkaline phosphatase (SEAP), and the interferon regulatory factor (IRF) pathway, by monitoring the activity of Lucia luciferase. The reporter proteins were measured quantitatively using QUANTI-Blue Solution, a SEAP detection reagent, and a Lucia and Gaussia luciferase detection reagent (QUANTI-Luc4 Lucia/Gaussia). 2,3-cyclic GMP-AMP (cGAMP) and LPS (100 ng/mL) were used as positive controls and fresh DMEM as a negative control. Two concentrations (100 M and 200 μM) of all HA polymers were prepared in Nanopure™ water and used in the assay. Activities were analyzed after 24 hours. For NF-κB activity, L of cell supernatant was mixed with 180 μL QUANTI-Blue solution in a 96-well plate, and the cells were incubated at 37° C. for 30 minutes. The absorbance was determined at 630 nm using a plate reader (Bio-Tek Synergy 2). For IRF3 activity, 20 μL of cell supernatant was mixed with 50 μL QUANTI-Luc4 Lucia/Gaussia solution in a 96-well plate, and luminescence was recorded using Bio-Tek Synergy 2 plate reader. Data was analyzed using GraphPad Prism.

### ELISA Analysis for Cytokine Release:

To analyze the effect of HA polymers on the release of IL-1β, TNF-α, and IL-6 from macrophages, an ELISA was performed. 100,000 J774-Dual macrophages/well were seeded using DMEM in a tissue culture-treated 96-well plate and incubated overnight at 37° C. with 5% CO₂ in a humidified incubator. On the same day, an ELISA plate was coated with capture antibody for IL-IP, TNF-α, or IL-6, according to the manufacturer's instructions, sealed with plate film, and incubated at room temperature overnight. The next day, cells were either left untreated or treated with 100 μM of HA, HAA, or HAD and incubated for 24 hours. ELISA plates were washed and blocked during the priming period according to the manufacturer's protocol. To assess IL-1β release, the cells were treated with ATP (5 mM final concentration) and incubated for 45 minutes. 100 μL cell supernatants from the treatment groups were collected, transferred to the blocked ELISA plate along with either IL-1β, TNF-α, or IL-6 standards, and incubated overnight at 4° C. The next day, the ELISA plate was washed, probed with detection antibodies, and developed according to the manufacturer's procedure. The developed ELISA plate was read on a Biotek Synergy 2 plate reader at 450 nm with background subtraction at 540 nm. Data analysis was performed in GraphPad Prism.

Uptake Studies with Keratinocytes and Macrophages:

Cellular uptake of rhodamine-conjugated hyaluronic acid polymers was imaged using a Nikon AXR confocal microscope. HaCaT cells (human keratinocytes) or J774-Dual macrophages were seeded at a density of 40,000-100,000 cells/well in 8-well ibidi slides and incubated for 24 hours before treatment. Cells were treated with HAR, HAAR, or HADR at a final drug concentration of 100 μM and imaged after 24 hours to visualize cellular uptake and subcellular distribution. HAR with the same concentration was used as the control for this experiment.

Calcium Flux Induction with HA Polymers:

An intracellular calcium flux assay was employed to check intracellular Ca2+ ion signal transduction pathways (Yaron, J. R., et al., 2015, Cell Death & Disease, 6(10):e1954; Yaron, J. R., et al., 2016, Biochemical and Biophysical Research Communications, 472(3):545-550). To investigate the effects of HA polymers on keratinocyte calcium ion signaling, 40,000 HaCaT cells were seeded in 8-well ibidi slides and incubated at 37° C. overnight. Calbryte™ 520 AM, a fluorescent, cell-permeable indicator, was used to measure intracellular calcium. The cells were treated with Calbryte™ 520 AM (2 μM) and, after preincubation at 47° C. for 30 minutes, Hoechst stain (1 μg/mL). For Ca²⁺ measurements, the cells were activated either with 100 μM histamine or HA polymers. For understanding the basic G protein-coupled receptors (GPCR)-mediated signaling pathway, the cells were treated with 100 μM histamine receptor inhibitors/antagonists (HRH1-4) in the culture medium for 30 min at 37° C. Calbryte™ 520 AM was excited with a 488 nm laser in wide-field mode to record global oscillations in cytosolic Ca2+ levels. In contrast, Hoechst stain was excited with a 402 nm laser using a Nikon AXR confocal microscope. The pattern of Ca²⁺ oscillations (F/F₀) was acquired from the images obtained at intervals of 3 s/frame. Quantification of Ca²⁺ signals was calculated based on the area under the curve.

In Scratch Assay with Keratinocytes:

HaCaT cells were seeded into 24-well plates and grown to a confluent layer in DMEM supplemented with 10% FBS and 1% penicillin/streptomycin. The following day, HaCaT cells were starved with serum-free media for 24 hours. Cell monolayers were carefully scratched with a sterile 200 μL pipette tip and washed twice with phosphate buffer saline (PBS). The scratches were treated with 1% FBS DMEM with or without 100 μM HA-HMW, HAA-HMW, or HAD-HMW, and incubated at 37° C. 1 ng/mL TGF-β was used as a positive control for this experiment. Reference points near the scratch were marked to ensure the same area was imaged. Images of wound gaps were taken at different time points (days 0, 1, and 2) using a high-resolution mobile camera mounted on a microscope eyepiece. The percentage of scratch closure was calculated using ImageJ. All scratch assays were performed in triplicate.

### Wound Healing Studies in BALB/c Mice:

The efficacy of hyaluronic acid derivatives in wound closure was examined using BALB/c mice (Yaron, J. R., et al., 2020, Pharmaceutics, 12(11):1003). 8-10-week-old BALB/c mice were obtained from The Jackson Laboratory. Mice were quarantined for at least 1 week before the procedure. Mice were anesthetized with a cocktail of 120 mg/kg ketamine and 6 mg/kg xylazine by intraperitoneal injection (100 μL/25 g bodyweight). The dorsal surface of BALB/c mice was shaved with a clipper and sterilized using chlorhexidine gluconate and 70% isopropyl alcohol with sterile cotton swabs. Eye lube was applied to the eyes. Full-thickness wounds were created using 5 mm biopsy punches (Integra Miltex, VWR). 20 μL of polymer solution or saline was applied topically on the wound bed, and wounds were splinted and covered with Tegaderm. Wound images were captured from day 0 until necropsy day.

### Histology and Immunohistochemistry:

Mice were euthanized on day 8 with CO₂, and wound tissues were collected and fixed in 10% neutral-buffered formalin. Tissues were dehydrated through graded alcohol into xylene and embedded in paraffin blocks. The tissue sections were placed onto positively charged glass slides. Sections were rehydrated with tap water, and epitope retrieval was performed with sodium citrate buffer (10 mM, pH 6.0, with 0.05% Tween-20®) at 60° C. overnight. Sections were blocked with 5% bovine serum albumin (BSA) in TBST (0.1% Tween-20) for 1-2 hours at room temperature and incubated with 5% BSA in TBST containing primary rabbit antibody markers at 4° C. overnight. Sections were washed with TBST, and peroxide quenching was performed using 3% hydrogen peroxide in 1λPBS for 15 minutes at room temperature. Sections were incubated in horseradish peroxidase (HRP)-conjugated donkey anti-rabbit secondary antibody (1:500) for 2 hours at room temperature, followed by thorough washing. The sections were developed with ImmPACT DAB substrate for 2-5 minutes, counterstained with hematoxylin, and mounted with CytoSeal XYL (Thermo Fisher). Slides were scanned at 20× magnification using an Olympus VS200 Slide Scanner and quantified with QuPath.

### Statistical Analysis:

Data are expressed as mean±standard error of mean. Statistical significance analyses were performed using either one-way or two-way analysis of variance (ANOVA). P-values<0.05 are considered statistically significant. All data were analyzed and plotted by GraphPad Prism Software.

Despite advances in acute wound dressings using biomaterials, the use of biocompatible polymers to prevent bacterial infection and achieve full-thickness wound closure remains suboptimal as a viable therapeutic wound-healing option. Here, hyaluronic acid-derived biomaterials, which promote the innate immune response and internalization into keratinocytes and macrophages, demonstrated enhanced full-thickness wound closure. Histamine- or histidine-conjugated hyaluronic acid biomaterials led to infiltration of pro-resolution macrophages and neutrophils into the wound bed, accelerating wound healing in mice, a significant effect compared with control groups.

For the first time, this study demonstrates that histamine- or histidine-conjugated hyaluronic acid biomaterials enhance wound healing and tissue regeneration in mouse models while increasing cellular uptake and the efficacy of calcium signaling pathways. Histamine- or histidine-conjugated HA polymers promote an enhanced innate, inflammatory, and neutrophilic immune response, as demonstrated by immunohistochemistry analysis. Further, HAA and HAD modulated immune cell activity in the wound bed, which had a more sustained reparative immune cell population during the late stages of healing. These biomaterials can be useful for tissue repair across a variety of wound types, including obese and diabetic wounds, as well as other skin diseases.

What is claimed is:

1. A composition comprising a polyvalent polymer, wherein the polyvalent polymer comprises at least one small molecule conjugated to a polymer backbone,
wherein the at least one small molecule is selected from the group consisting of histamine, histidine, and variants and derivatives thereof.

2. The composition of claim 1, wherein the polymer backbone is a hyaluronic acid (HA) backbone.

3. The composition of claim 2, wherein between about 25% and about 35% of all disaccharide monomers in the HA backbone are conjugated to a small molecule.

4. The composition of claim 2, wherein the HA is a high-molecular-weight (HMW) HA.

5. The composition of claim 4, wherein the HMW HA has:
a) an average molecular weight between about 1 and about 1.5 MDa;
b) a polydispersity index of less than about 1;
c) a hydrodynamic diameter between about 1,200 nm and about 1,500 nm; and/or
d) a zeta potential between about −11 mV and about −14 mV.

6. The composition of claim 1, wherein the composition comprises an aqueous solution of the polyvalent polymer in a concentration of between about 20 mg/mL and about 100 mg/mL.

7. The composition of claim 1, wherein the composition is a gel or a hydrogel.

8. The composition of claim 1, wherein the composition further comprises at least one additional agent.

9. The composition of claim 8, wherein the at least one additional agent is selected from the group consisting of an antibiotic agent, an antiviral agent, an antifungal agent, an antiparasitic agent, an analgesic agent, an anesthetic agent, an anti-inflammasome agent, a pro-healing bioactive, an antioxidant, and/or a combination thereof.

10. A functionalized wound dressing comprising a wound dressing saturated or impregnated with the composition of claim 1, wherein, when applied to a wound, the functionalized wound dressing releases the polyvalent polymer into the wound.

11. The functionalized wound dressing of claim 10, wherein the wound dressing is in the form of a bandage, gauze, or patch.

12. The functionalized wound dressing of claim 11, wherein the bandage, gauze, or patch is biodegradable.

13. The functionalized wound dressing of claim 10, wherein the wound dressing comprises a material selected from the group consisting of a natural polymer and a synthetic polymer.

14. The functionalized wound dressing of claim 13, wherein:
a) the natural polymer is at least one selected from the group consisting of cellulose, starch, pectin, gelatin, chitosan, collagen, latex, alginate, carrageenan, silk fibroin, keratin, and/or a combination thereof; or
b) the synthetic polymer is selected form the group consisting of polyethylene (PE), polysulfone (PS), polypropylene (PP), polylactic acid (PLA), poly(lactide-co-glycolide) (PLGA), polyglycoliyc acid (PGA), polycaprolactone (PCL), polyvinyl alcohol (PVA), polyvinyl chloride (PVC), polyurethane, polyvinylpyrrolidone (PVP), ethyl cellulose, methyl cellulose, nitrocellulose, poly(methacrylate-isobutene-monoisopropylmaleaste), polytetrafluoroethylene (PTFE), polyvinylidene fluoride (PVF), polyacrylate, and/or a combination thereof.

15. The composition of claim 8, wherein the composition is a vaccine composition, wherein the at least one additional agent comprises at least one antigenic small molecule, glycan, protein, nucleic acid, virus, fragment of a virus, organism, fragment of an organism, or combination thereof.

16. A method of treating a wound in a tissue comprising:
a) applying to the wound:
i) the composition of claim 1 and a wound dressing; or
ii) a functionalized wound dressing comprising a wound dressing saturated or impregnated with the composition of claim 1, wherein, when applied to a wound, the functionalized wound dressing releases the polyvalent polymer into the wound; and
b) allowing the wound to heal with the immunomodulatory agent and wound dressing.

17. The method of claim 16, further comprising using a suture, staple, clamp, adhesive, or other structural element with the wound dressing or functionalized wound dressing to fasten the wound dressing or functionalized wound dressing to the tissue.

18. The method of claim 16, further comprising:
removing the wound dressing or functionalized wound dressing from the wound; and
applying a second wound dressing to the wound with or without applying a second administration of the composition; or
applying a second functionalized wound dressing to the wound.

19. The method of claim 16, wherein the wound is an acute wound or a diabetic wound.

20. A method of inducing an immune response in a subject comprising administering to the subject the composition of claim 15.
